(19) United States
(12) Patent Application Publication
(10) Pub. No.: US 2022/0288131 A1
(43) Pub. Date: Sep. 15, 2022

(54) COMPOSITIONS AND METHODS OF TREATING VASCULAR DISEASES

(71) Applicant: Astellas Institute for Regenerative Medicine, Westborough, MA (US)

(72) Inventors: Maria Mirotsou, Westborough, MA (US); Nutan Prasain, Westborough, MA (US); Amrita Singh, Westborough, MA (US); Robert Lanza, Westborough, MA (US)

(21) Appl. No.: 17/632,723

(22) PCT Filed: Aug. 27, 2020
(86) PCT No.: PCT/US2020/048076

§ 371 Date: Feb. 3, 2022

Related U.S. Application Data
(60) Provisional application No. 62/892,724, filed on Aug. 28, 2019.

(51) Int. Cl. A61K 35/44; A61K 47/26; A61K 47/02; A61P 9/14; C12N 5/071

(52) U.S. Cl. CPC A61K 35/44; A61K 47/26; A61K 47/02; A61P 9/14; C12N 5/0692; C12N 2501/165; C12N 2501/115; C12N 2501/155; C12N 2501/15; C12N 2506/45; C12N 2501/16; C12N 2533/90; C12N 2533/54

(57) ABSTRACT

The present invention generally relates to novel mesoderm-derived vascular progenitor cells (meso-VPCs) and methods of producing the meso-VPCs. The present invention also relates to methods of treating a vascular disease, such as critical limb ischemia, by administering the meso-VPCs into a subject.

### RELATED APPLICATIONS

This application claims the benefit under 35 U.S.C. § 119(e) of U.S. Provisional Application No. 62/892,724, filed Aug. 28, 2019, which is hereby incorporated by reference in its entirety.

### FIELD OF THE INVENTION

The instant invention relates to novel mesoderm-derived vascular progenitor cells (meso-VPCs) and methods of producing meso-VPCs. The instant invention also relates to methods of treating a vascular disease, such as ischemia, using the meso-VPCs.

### BACKGROUND OF THE INVENTION

Vascular diseases are conditions that affect the body's network of blood vessels. More than 78 million Americans have the most common form of vascular disease, high blood pressure. In addition, peripheral artery disease (PAD) affects 12-15 million people in the United States, with a much larger number of undiagonosed cases.

Peripheral artery disease (PAD) is the narrowing or blockage of the vessels that carry blood from the heart to other organs and tissues. It is primarily caused by the buildup of fatty plaque in the arteries, which is called atherosclerosis. PAD can occur in any blood vessel, but it is more common in the legs than the arms.

Ischemia is a condition caused by peripheral artery disease involving an interruption in the arterial blood supply to a tissue, organ, or extremity that, if untreated, can lead to tissue death. It can be caused by embolism, thrombosis of an atherosclerotic artery, or trauma. Venous problems like venous outflow obstruction and low-flow states can cause acute arterial ischemia. Ischemia in the legs can lead to leg pain or cramps with activity (claudication), changes in skin color, sores or ulcers and feeling tired in the legs. Total loss of circulation can lead to gangrene and loss of a limb.

Treatment for vascular diseases such as ischemia is limited. While most of the treatment methods involve invasive surgical procedures, the others focus on the prevention of progression of existing conditions. Accordingly, there is still a need in the art for improved treatments for vascular diseases such as ischemia.

### SUMMARY OF THE INVENTION

The present invention relates to novel methods of producing mesoderm-derived vascular progenitor cells (meso-VPCs) by in vitro differentiation of pluripotent stem cells. The present invention further provides methods of treating vascular diseases, e.g., critical limb ischemia, using the meso-VPCs of the current invention.

Accordingly, in one aspect, the present invention provides a method of producing a population of mesoderm-derived vascular progenitor cells (meso-VPCs) from a pluripotent stem cell, wherein the method comprises culturing a mesoderm cell derived from a pluripotent stem cell under non-adherent or low adherent conditions, in a medium comprising one or more factors selected from the group consisting of vascular endothelial growth factor (VEGF), fibroblast growth factor (FGF), bone morphogenetic protein 4 (BMP4), and a small molecule inhibitor of transforming growth factor-beta (TGF-β) type I receptor, thereby producing a population of mesoderm-derived vascular progenitor cells (meso-VPCs).

In one embodiment, the mesoderm cell is derived from a pluripotent stem cell by culturing the pluripotent stem cell in a medium comprising one or more mesoderm inducing growth factors selected from the group consisting of Activin-A, vascular endothelial growth factor (VEGF), fibroblast growth factor (FGF), and bone morphogenetic protein 4 (BMP4).

In one embodiment, the meso-VPCS are produced as a vasculonoid. In another embodiment, the meso-VPCs are dissociated into single cells.

In one embodiment, the mesoderm inducing growth factors comprise Activin-A, VEGF165, FGF-2 and BMP4. In one embodiment, the Activin-A is used at a concentration of about 5-15 ng/mL. In one embodiment, the VEGF165 is used at a concentration of about 5-25 ng/mL. In one embodiment, the FGF-2 is used at a concentration of about 5-25 ng/mL. In one embodiment, the BMP4 is used at a concentration of about 5-50 ng/mL. In one embodiment, the method further comprises removing Activin-A from the culture media after about 24 hours of culturing.

In one embodiment, the pluripotent stem cells are cultured on an extracellular matrix surface. In one embodiment, the extracellular matrix surface is a Matrigel-coated surface. In one embodiment, the pluripotent stem cells are cultured for about 3 days to about 5 days.

In one embodiment, the small molecule inhibitor of transforming growth factor-beta (TGF-β) type I receptor is SB431542. In another embodiment, the one or more factors comprise VEGF165, FGF-2, BMP4, and SB431542. In one embodiment, the one or more factors further comprises Forskolin. In one embodiment, the Forskolin is used at a concentration of about 2-10 μM. In one embodiment, the VEGF165 is used at a concentration of about 10-50 ng/mL. In one embodiment, the FGF-2 is used at a concentration of about 10-50 ng/mL. In one embodiment, the BMP4 is used at a concentration of about 10-50 ng/mL. In one embodiment, the SB431542 is used at a concentration of about 5-20 μM.

In one embodiment, culturing the mesoderm cell is performed for about 3 days to about 7 days.

In one embodiment, culturing the mesoderm cell is conducted under a normoxia condition of 5% CO₂ and 20% O₂.

In one embodiment, culturing of the pluripotent stem cells is conducted under a normoxia condition of 5% CO₂ and 20% O₂.

In one embodiment, the non-adherent or low adherent conditions are on an ultra-low attachment surface.

In one aspect, the present invention provides a method of producing a population of mesoderm-derived vascular progenitor cell (meso-VPC) from a pluripotent stem cell, wherein the method comprises (a) culturing a mesoderm cell derived from a pluripotent stem cell on an extracellular matrix surface, in a medium comprising one or more factors selected from the group consisting of vascular endothelial growth factor (VEGF), fibroblast growth factor (FGF), and bone morphogenetic protein 4 (BMP4); and (b) culturing the cells produced in step (a) on an extracellular matrix surface, in a medium comprising one or more factors selected from the group consisting of vascular endothelial growth factor (VEGF), fibroblast growth factor (FGF), bone morphogenetic protein 4 (BMP4), and a small molecule inhibitor of transforming growth factor-beta (TGF-β) type I receptor, thereby producing the population of mesoderm-derived vascular progenitor cells.

In one embodiment, the mesoderm cell is derived from a pluripotent stem cell by culturing the pluripotent stem cell in a medium comprising one or more mesoderm inducing growth factors selected from the group consisting of Activin-A, vascular endothelial growth factor (VEGF), fibroblast growth factor (FGF), and bone morphogenetic protein 4 (BMP4).

In one embodiment, the method further comprises dissociating the population of meso-VPCs into single cells.

In one embodiment, the mesoderm inducing growth factors comprise Activin-A, VEGF165, FGF-2 and BMP4. In one embodiment, the Activin-A is used at a concentration of about 5-15 ng/mL. In one embodiment, the VEGF165 is used at a concentration of about 5-25 ng/mL. In one embodiment, the FGF-2 is used at a concentration of about 5-25 ng/mL. In one embodiment, the BMP4 is used at a concentration of about 5-50 ng/mL. In one embodiment, the method further comprises removing Activin-A from the culture media after about 24 hours of culturing.

In one embodiment, the extracellular matrix surface in step (a) is a collagen IV-coated surface.

In one embodiment, the pluripotent stem cells are cultured for about 3 days to about 5 days.

In one embodiment, the one or more factors in step (a) comprise VEGF165, FGF-2, and BMP4.

In one embodiment, the small molecule inhibitor of transforming growth factor-beta (TGF-β) type I receptor is SB431542. In another embodiment, the one or more factors in step (b) comprise VEGF165, FGF-2, BMP4, and SB431542.

In one embodiment, the one or more factors in step (a) further comprises Forskolin.

In one embodiment, the one or more factors in step (b) further comprises Forskolin.

In one embodiment, the Forskolin is used at a concentration of about 2-10 μM.

In one embodiment, the VEGF165 is used at a concentration of about 10-50 ng/mL.

In one embodiment, the FGF-2 is used at a concentration of about 10-50 ng/mL.

In one embodiment, the BMP4 is used at a concentration of about 10-50 ng/mL.

In one embodiment, the SB431542 is used at a concentration of about 5-20 μM.

In one embodiment, the extracellular matrix surface in steps (a) and (b) is a collagen-IV-coated surface.

In one embodiment, the culturing in step (a) is performed for about 1 day.

In one embodiment, the culturing in step (b) is performed for about 4 days to about 7 days.

In one embodiment, the culturing in step (a) is conducted under a normoxia condition of 5% CO₂ and 20% O₂.

In one embodiment, the culturing in step (b) is conducted under a hypoxia condition of 5% CO₂ and 5% O₂.

In one embodiment, culturing of the pluripotent stem cells is conducted under a normoxia condition of 5% CO₂ and 20% O₂.

In one embodiment, the pluripotent stem cell is a human embryonic stem cell.

In one embodiment, the pluripotent stem cell is a human induced pluripotent stem cell.

In one embodiment, the population of meso-VPCs produced according to any of the methods of the present invention expresses at least one of the cell-surface markers selected from the group consisting of CD31/PECAM1, CD309/KDR, CD43, CD144, CD34, CD184/CXCR4, CD146, and PDGFRb.

In one embodiment, the population of meso-VPCs produced according to any of the methods of the present invention expresses cell-surface markers (a) CD146, CD31/PECAM1, and CD309/KDR; or (b) CD31/PECAM1, CD309/KDR, CD146, and (i) at least one of CD144, CD34, CD184/CXCR4, CD43, or PDGFRb, (ii) CD34, CD184/CXCR4, and PDGFRb; (iii) CD184/CXCR4; (iv) PDGFRb; (v) CD144 and CD184/CXCR4; (vi) CD184/CXCR4 and CD43; or (vii) CC184/CXCFR4.

In one embodiment, the population of meso-VPCs produced according to any of the methods of the present invention exhibits limited or no detection of (a) one or more of cell-surface markers selected from the group consisting of CXCR7, CD45, and NG2; (b) CXCR7, CD45, and NG2; or (c) one or more of cell-surface markers selected from the group consisting of CD144, CD34, CD184/CXCR4, CXCR7, CD43, CD45, PDGFRb, and NG2.

In one embodiment, the population of meso-VPCs produced according to any of the methods of the present invention expresses at least one miRNA marker selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p.

In one embodiment, the population of meso-VPCs produced according to any of the methods of the present invention exhibits limited or no expression of at least one miRNA marker selected from hsa-let-7e-3p, hsa-miR-99a-3p, hsa-miR-133a-5p, hsa-miR-11399, hsa-miR-196b-3p, hsa-miR-5690, and hsa-miR-7151-3p.

In one embodiment, the population of meso-VPCs produced according to any of the methods of the present invention expresses hsa-miR-3917, hsa-miR-450a-2-3p, and hsa-miR-542-5p.

In one embodiment, the population of meso-VPCs produced according to any of the methods of the present invention comprises at least one meso-VPC positive for at least one miRNA markers selected from the group consisting of mir126, mir125a-5p, mir24, and mir483-5p. In one embodiment, the miRNA marker is mir483-5p.

In one embodiment, the population of meso-VPCs produced according to any of the methods of the present invention comprises at least one meso-VPC that exhibits limited or no expression for at least one miRNA markers selected from the group consisting of mir367, mir302a, mir302b, mir302c, mirLet7-e, mir223, mir99a, mir142-3p, and mir133a.

In one embodiment, the methods of the present invention further comprise producing a vascular endothelial cell by differentiation of the meso-VPC.

In one embodiment, the differentiation is performed on a fibronectin-coated surface.

In one aspect, the present invention provides a composition comprising a population of meso-VPCs produced by any one of the methods of the invention.

In one aspect, the present invention provides a composition comprising a population of mesoderm-derived vascular progenitor cells (meso-VPCs) produced by in vitro differentiation of a mesoderm cell derived from a pluripotent stem cell, wherein the population of meso-VPCs expresses at least one cell-surface marker selected from the group consisting of CD31/PECAM1, CD309/KDR, CD43, CD144, CD34, CD184/CXCR4, CD146, and PDGFRb.

In one embodiment, the composition comprising a population of meso-VPCs expresses at least two cell-surface markers selected from the group consisting of CD31/PECAM1, CD309/KDR, CD43, CD144, CD34, CD184/CXCR4, CD146, and PDGFRb.

In one embodiment, the composition comprising a population of meso-VPCs expresses cell surface markers CD146, CD31/PECAM1, and CD309/KDR.

In one embodiment, the composition comprising a population of meso-VPCs expresses cell surface markers CD31/PECAM1, CD309/KDR, CD146, and (i) at least one of CD144, CD34, CD184/CXCR4, CD43, or PDGFRb, (ii) CD34, CD184/CXCR4, and PDGFRb; (iii) CD184/CXCR4; (iv) PDGFRb; (v) CD144 and CD184/CXCR4; (vi) CD184/CXCR4 and CD43; or (vii) CC184/CXCFR4.

In one embodiment, the composition comprising a population of meso-VPCs exhibits limited or no detection of (a) one or more cell surface markers selected from the group consisting of CXCR7, CD45, and NG2; (b) CXCR7, CD45, and NG2; or (c) one or more cell surface markers selected from the group consisting of CD144, CD34, CD184/CXCR4, CXCR7, CD43, CD45, PDGFRb, and NG2.

In one embodiment, the composition comprising a population of meso-VPCs expresses at least one miRNA marker selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p.

In one embodiment, the composition comprising a population of meso-VPCs exhibits limited or no expression of at least one miRNA marker selected from hsa-let-7e-3p, hsa-miR-99a-3p, hsa-miR-133a-5p, hsa-miR-11399, hsa-miR-196b-3p, hsa-miR-5690, and hsa-miR-7151-3p.

In one embodiment, the composition comprising a population of meso-VPCs expresses hsa-miR-3917, hsa-miR-450a-2-3p, and hsa-miR-542-5p.

In one embodiment, the population of meso-VPCs comprises vasculonoids of meso-VPCs.

In one embodiment, the population of meso-VPCs comprises single cells of meso-VPCs.

In one embodiment, the present invention provides a meso-VPC produced by in vitro differentiation of a mesoderm cell derived from a pluripotent stem cell, wherein the meso-VPC is positive for at least one miRNA marker selected from the group consisting of mir126, mir125a-5p, mir24, and mir483-5p.

In one embodiment, the meso-VPC is positive for miRNA marker mir483-5p.

In one embodiment, the meso-VPC is negative for at least one miRNA marker selected from the group consisting of mir367, mir302a, mir302b, mir302c, mirLet7-e, mir223, mir99a, mir142-3p, and mir133a.

In one embodiment, the pluripotent stem cell is a human pluripotent stem cell.

In one embodiment, the pluripotent stem cell is human embryonic stem cell (hESC).

In one embodiment, the pluripotent stem cell is human induced pluripotent stem cell (hiPSC).

In one embodiment, the pluripotent stem cell is first differentiated into a mesoderm cell which, in turn, is differentiated into the meso-VPC.

In one aspect, the present invention provides a pharmaceutical composition comprising a composition comprising a population of meso-VPCs or any one of the meso-VPCs of the present invention.

In one aspect, the present invention provides a method of treating a vascular disease or disorder in a subject, the method comprising administering to the subject an effective amount of any one of the compositions comprising a population of meso-VPCs or mesoderm-derived vascular progenitor cells (meso-VPCs) of the present invention, or any one of the pharmaceutical compositions of the present invention, thereby treating the vascular disease or disorder in the subject.

In one embodiment, the vascular disease or disorder is selected from the group consisting of atherosclerosis, peripheral artery disease (PAD), carotid artery disease, venous disease, blood clots, aortic aneurysm, fibromuscular dysplasia, lymphedema, and vascular injury.

In one embodiment, the peripheral artery disease is selected from the group consisting of critical limb ischemia, intestinal ischemic syndrome, renal artery disease, popliteal entrapment syndrome, Raynaud's phenomenon, Buerger's disease.

In one embodiment, the periphery artery disease is critical limb ischemia.

In one embodiment, the composition comprising a population of meso-VPCs, meso-VPC, or the pharmaceutical composition is administered intramuscularly or systemically.

In one embodiment, the administration of the composition comprising a population of meso-VPCs, meso-VPC, or the pharmaceutical composition increases the blood flow in the subject.

In one embodiment, the administration of the composition comprising a population of meso-VPCs, meso-VPC, or the pharmaceutical composition promotes the angiogenesis and/or vasculogenesis in the subject.

In one embodiment, the administration of the composition comprising a population of meso-VPCs, meso-VPC, or the pharmaceutical composition reduces the ischemic severity in the subject.

In one embodiment, the administration of the composition comprising a population of meso-VPCs, meso-VPC, or the pharmaceutical composition reduces the necrosis area of the limb in the subject.

In one embodiment, about 1×10⁴ to about 1×10¹³ meso-VPCs are administered to the subject.

In one embodiment, the meso-VPC is administered in a pharmaceutical composition.

In one embodiment, the pharmaceutical composition comprises (a) a buffer, maintaining the solution at a physiological pH; (b) at least 5% (w/v) glucose; and (c) an osmotically active agent maintaining the solution at a physiologically osmolality.

In one embodiment, the glucose is D-glucose (Dextrose).

In one embodiment, the osmotically active agent is a salt.

In one embodiment, the salt is sodium chloride.

### BRIEF DESCRIPTION OF THE DRAWINGS

FIG. 1 is a schematic illustration of the process for the in vitro differentiation of human pluripotent stem cells into mesoderm cells.

FIG. 2A is a graph showing expression of cell-surface markers KDR, CD56/NCAM1, APLNR/APJ, GARP, or CD13 on mesoderm cells differentiated from human induced pluripotent stem cell line GMP1, confirming differentiation to the mesoderm lineage.

FIG. 2B is a graph showing limited or no expression of pluripotent, endoderm, ectoderm, and hematovascular cell-surface markers on mesoderm cells differentiated from human induced pluripotent stem cell line GMP1, confirming the differentiation to the mesoderm lineage.

FIG. 3 is a schematic illustration of the process for the in vitro differentiation of human pluripotent stem cells into mesoderm cells (left), and the in vitro differentiation of mesoderm cells into mesoderm-derived vascular progenitor cells (meso-VPCs) using the Meso-3D-Vasculonoid VPC1 protocol (upper right), or the Meso-3D-Vasculonoid VPC2 protocol (lower right).

FIG. 4 is a schematic illustration of the process for the in vitro differentiation of human pluripotent stem cells into mesoderm cells (left), and the in vitro differentiation of mesoderm cells into mesoderm-derived vascular progenitor cells (meso-VPCs) using the Meso-2D VPC2 protocol (upper right), or the Meso-2D VPC3 protocol (lower right).

FIG. 5 is a panel of microscopic images showing the capacity of meso-VPCs produced by Meso-3D-Vasculonoid protocols to undergo further differentiation into the endothelial lineage. The upper panel shows the morphology of meso-VPCs at Day 5 prior to harvest. The middle panel shows endothelial differentiation of meso-VPCs using fibronectin-coated plates and medium that promotes endothelial differentiation. The lower panel shows the capillary-like Matrigel networks formed by the meso-VPCs.

FIG. 6A is a graph showing expression of cell-surface markers CD31/PECAM1, CD309/KDR, CXCR4/CD184, CD43, CD146, and PDGFRb on meso-VPCs produced using Meso-3D-Vasculonoid-VPC1, Meso-3D-Vasculonoid-VPC2, Meso-2D-VPC2, or Meso-2D-VPC3 protocols.

FIG. 6B is a heat-map showing fractions of meso-VPCs and comparative hemogenic endothelial cells (HE) or hemangioblasts (HB) that are positive for selected cell-surface markers. Comparisons with undifferentiated pluripotent stem cells (J1 and GMP1) and human umbilical vein endothelial cells (HUVECs) cells are also shown.

FIG. 6C is a principal component analysis (PCA) plot showing vascular cell-surface marker expression profiles of meso-VPCs produced by Meso-3D-Vasculonoid protocols or Meso-2D protocols, comparative hemogenic endothelial cells (HE), comparative hemangioblasts (HB), undifferentiated pluripotent stem cells (J1 and GMP1) or human umbilical vein endothelial cells (HUVECs).

FIG. 7 is a panel of microscopic images showing the capacity of meso-VPCs produced by Meso-2D protocols to undergo further differentiation into endothelial lineage. The upper panel shows the morphology of meso-VPCs at Day 7 prior to harvest. The middle panel shows endothelial differentiation of meso-VPCs using fibronectin-coated plates and medium that promotes endothelial differentiation. The lower panel shows the capillary-like Matrigel networks formed by the meso-VPCs.

FIG. 8 is a graph showing increased blood flow in animals treated with meso-VPCs as described in Example 9. Specifically, animals are sham-operated (1M), or treated with vehicle control (2M), J1-HDF Meso-2D VPC2 (3M), J-HDF Meso-3D Vasculonoid VPC2 (4M), GMP1HDF Meso-2D VPC2 (5M), GMP1-HDF Meso-3D Vasculonoid VPC2 (6M) or GMP1-HDF Meso-3D Vasculonoid VPC1 (7M).

FIG. 9 is a graph showing changes in blood vessel density in animals treated with meso-VPCs as described in Example 9. Specifically, animals are treated with vehicle control (2M), J1-HDF Meso-2D VPC2 (3M TI1), J-HDF Meso-3D Vasculonoid VPC2 (4M TI2), GMP1HDF Meso-2D VPC2 (5M TI3), GMP1-HDF Meso-3D Vasculonoid VPC2 (6M TI4) or GMP1-HDF Meso-3D Vasculonoid VPC1 (7M TI5).

FIG. 10 is a graph showing combined quantitative results of CD34⁺ staining, which is an indication for small capillaries formation, total vessel numbers, and blood flow test in animals treated with meso-VPCs. Specifically, animals are sham-operated (1M), or treated with vehicle control (2M), J1-HDF Meso-2D VPC2 (3M TI1), J-HDF Meso-3D Vasculonoid VPC2 (4M TI2), GMP1HDF Meso-2D VPC2 (5M TI3), GMP1-HDF Meso-3D Vasculonoid VPC2 (6M TI4) or GMP1-HDF Meso-3D Vasculonoid VPC1 (7M TI5).

FIG. 11 shows strong and statistically significant correlation between blood flow measured by Laser Doppler and average capillaries density of each group of animals treated with meso-VPCs.

FIG. 12A provides a plot and graph that show unique human miRNAs found in the population of J1-derived Meso-3D Vasculonoid VPC2 cells from three replicates, including hsa-miR-3917, hsa-miR-450a-2-3p, and hsa-miR-542-5p, as compared to the population of J1 cells and population of J1-derived HE cells. FIG. 12A also shows unique human miRNAs found in the population of J1-derived HE cells, including hsa-miR-11399, hsa-miR-196b-3p, hsa-miR-5690, and hsa-miR-7151-3p. “Expressed” is normalized expression >0 in all 3 replicates.

FIG. 12B is a graph showing expression levels of miRNAs in the population of J1-derived Meso-3D Vasculonoid VPC2 cells that were previously analyzed on single cells and shows that hsa-miR-126-5p, hsa-miR-125a-5p, and hsa-miR-24-3p are expressed in both the population of J1 cells and the population of J1-derived Meso-3D Vasculonoid VPC2 cells.

FIG. 12C is a graph showing that the population of J1-derived Meso-3D Vasculonoid VPC2 cells expresses hsa-let-7e-5p, hsa-miR-99a-5p, hsa-miR-223-5p, and hsa-miR-142-3p and does not express or has low expression of hsa-let-7e-3p, hsa-miR-99a-3p, and hsa-miR-133a-5p.

FIG. 12D is a graph that shows that the population of J1-derived Meso-3D Vasculonoid VPC2 cells express hsa-miR-483-5p and hsa-miR-483-3p.

FIG. 13 is a graph that shows the expression of the genes most up- or down-regulated in J1-derived Meso-3D Vasculonoid VPC2 cell sample as compared to single J1 or HUVEC cells in a single cell RNA-seq analysis.

FIG. 14A is an image at low magnification (10× objective) showing extensive vascular networks extending from the embedded aggregates of J1-derived Meso-3D Vasculonoid VPC2 vasculonoids by DAPI and UAE1 staining after 14 days.

FIG. 14B are graphs showing that when the J1-derived Meso-3D Vasculonoid VPC2 vasculonoids (“plural”) or J1-derived Meso-3D Vasculonoid VPC2 cells dissociated into single cells (“single cell”) were cultured in CLI-mimicking conditions in vitro under normoxia (20% O₂) (left panel) or hypoxia (5% O₂) (right panel) after thawing, the vasculonoids showed better cell survival compared to J1-derived Meso-3D Vasculonoid VPC2 cells that had been cryopreserved as single cells.

FIG. 14C is a graph that shows a statistically significant improvement in blood flow after administration of J1-derived Meso-3D Vasculonoid VPC2 single cells (“sc”) or vasculonoids throughout the study compared to vehicle treated group (GS2 media only); two-way ANOVA followed by Tukey's test.

FIG. 15A are graphs showing that animals treated with the meso-3D vasculonoid VPC2 cells had better average necrosis (left panel) and functional scores (right panel) at Day 21 compared to HE and HB cells. One-way ANOVA followed by Dunnett's test. Mean+/−sem.

FIG. 15B is a graph showing blood flow improvement at Day 63 in animals treated with the meso-3D vasculonoid VPC2 cells, HE, and HB cells, as compared to vehicle. *p<0.05 vs. vehicle. Mean+/−s.d. Two-way ANOVA followed by Tukey's test.

FIG. 15C is a graph showing CD34⁺ vessel growth in the quadriceps of animals treated with Meso-3D vasculonoid VPC2 cells, HE, and HB cells. *p<0.05 vs. vehicle. Mean+/−sem. Two-way ANOVA followed by uncorrected Fisher's LSD test.

FIG. 15D is a graph showing improvement in the grastrocnemius after administration of Meso-3D vasculonoid VPC2 cells, HE, or HB cells. *p<0.05 vs. vehicle. Mean+/−sem. Two-way ANOVA followed by uncorrected Fisher's LSD test.

FIG. 16A is a graph showing engrafted donor GMP1-Meso3D vasculonoid VPC2 cells by Ku80+ staining at Days 63 and 180 after treatment, indicating long-term engraftment of the cells.

FIG. 16B is a graph showing that by Days 35 and 63, the meso-3D vasculonoid VPC2 cells showed engraftment by Ku80+ staining.

FIG. 16C are fluorescence images of injected Meso3D vasculonoid VPC2s displaying long-term engraftment (Ku80+), formation of human vasculature (UEA1+ vessels), and promotion of paracrine host vessel growth (IB4+ and SMA+ vessels) 63 days after HLI surgery in Balb/c nude mice.

### DETAILED DESCRIPTION OF THE INVENTION

### I. Definitions

In order that the present invention may be more readily understood, certain terms are first defined. It should also be noted that whenever a value or range of values of a parameter are recited, it is intended that values and ranges intermediate to the recited values are also part of this invention.

In the following description, for purposes of explanation, specific numbers, materials, and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” mean that a particular feature, structure, or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” refers to one element or more than one element.

The term “comprising” or “comprises” is used herein in reference to compositions, methods, and respective component(s) thereof, that are essential to the disclosure, yet open to the inclusion of unspecified elements, whether essential or not.

“Pluripotent cells”, “pluripotent stem cells,” and “PSCs” as used herein, refer broadly to a cell capable of prolonged or virtually indefinite proliferation in vitro while retaining their undifferentiated state, exhibiting a stable (preferably normal) karyotype, and having the capacity to differentiate into all three germ layers (i.e., ectoderm, mesoderm and endoderm) under the appropriate conditions. Typically pluripotent cells (a) are capable of inducing teratomas when transplanted in immunodeficient (SCID) mice; (b) are capable of differentiating to cell types of all three germ layers (e.g., ectodermal, mesodermal, and endodermal cell types); and (c) express at least one hES cell marker (such as Oct-4, alkaline phosphatase, SSEA 3 surface antigen, SSEA 4 surface antigen, NANOG, TRA 1 60, TRA 1 81, SOX2, REX1). Exemplary pluripotent cells may express Oct-4, alkaline phosphatase, SSEA 3 surface antigen, SSEA 4 surface antigen, TRA 1 60, and/or TRA 1 81. Additional exemplary pluripotent cells include but are not limited to embryonic stem cells, induced pluripotent cells (iPS) cells, embryo-derived cells, pluripotent cells produced from embryonic germ (EG) cells (e.g., by culturing in the presence of FGF-2, LIF and SCF), parthenogenetic ES cells, ES cells produced from cultured inner cell mass cells (ICM), ES cells produced from a blastomere, and ES cells produced by nuclear transfer (e.g., a somatic cell nucleus transferred into a recipient oocyte). Exemplary pluripotent cells may be produced without destruction of an embryo. For example, induced pluripotent cells may be produced from cells obtained without embryo destruction. As a further example, pluripotent cells may be produced from a biopsied blastomere (which can be accomplished without harm to the remaining embryo); optionally, the remaining embryo may be cryopreserved, cultured, and/or implanted into a suitable host. Pluripotent cells (from whatever source) may be genetically modified or otherwise modified.

“Embryo” or “embryonic,” as used herein, refers broadly to a developing cell mass that has not implanted into the uterine membrane of a maternal host. An “embryonic cell” is a cell isolated from or contained in an embryo. This also includes blastomeres, obtained as early as the two-cell stage, and aggregated blastomeres.

“Embryonic stem cells” (ES cells or ESC) encompasses pluripotent cells produced from embryonic cells (such as from cultured inner cell mass cells or cultured blastomeres). Frequently such cells are or have been serially passaged as cell lines. Embryonic stem cells may be used as a pluripotent stem cell in the processes of producing mesoderm cells and meso-VPCs as described herein. For example, ES cells may be produced by methods known in the art including derivation from an embryo produced by any method (including by sexual or asexual means) such as fertilization of an egg cell with sperm or sperm DNA, nuclear transfer (including somatic cell nuclear transfer), or parthenogenesis. As a further example, embryonic stem cells also include cells produced by somatic cell nuclear transfer, even when non-embryonic cells are used in the process. For example, ES cells may be derived from the ICM of blastocyst stage embryos, as well as embryonic stem cells derived from one or more blastomeres. Such embryonic stem cells can be generated from embryonic material produced by fertilization or by asexual means, including somatic cell nuclear transfer (SCNT), parthenogenesis, and androgenesis. As further discussed above, ES cells may be genetically modified or otherwise modified.

ES cells may be generated with homozygosity or heterozygosity in one or more HLA genes, e.g., through genetic manipulation, screening for spontaneous loss of heterozygosity, etc. Embryonic stem cells, regardless of their source or the particular method used to produce them, typically possess one or more of the following attributes: (i) the ability to differentiate into cells of all three germ layers, (ii) expression of at least Oct-4 and alkaline phosphatase, and (iii) the ability to produce teratomas when transplanted into immunocompromised animals. Embryonic stem cells that may be used in embodiments of the present invention include, but are not limited to, human ES cells (“hESC” or “hES cells”) such as CT2, MA01, MA09, ACT-4, No. 3, J1, H1, H7, H9, H14 and ACT30 embryonic stem cells. Additional exemplary cell lines include NED1, NED2, NED3, NED4, NED5, and NED7. See also NIH Human Embryonic Stem Cell Registry. An exemplary human embryonic stem cell line that may be used is J1 cells.

Exemplary human embryonic stem cell (hESC) markers include, but are not limited to, alkaline phosphatase, Oct-4, Nanog, Stage-specific embryonic antigen-3 (SSEA-3), Stage-specific embryonic antigen-4 (SSEA-4), TRA-1-60, TRA-1-81, TRA-2-49/6E, Sox2, growth and differentiation factor 3 (GDF3), reduced expression 1 (REX1), fibroblast growth factor 4 (FGF4), embryonic cell-specific gene 1 (ESG1), developmental pluripotency-associated 2 (DPPA2), DPPA4, telomerase reverse transcriptase (hTERT), SALL4, E-CADHERIN, Cluster designation 30 (CD30), Cripto (TDGF-1), GCTM-2, Genesis, Germ cell nuclear factor, and Stem cell factor (SCF or c-Kit ligand). Additionally, embryonic stem cells may express Oct-4, alkaline phosphatase, SSEA 3 surface antigen, SSEA 4 surface antigen, TRA 1 60, and/or TRA 1 81.

The ESCs may be initially cultured in any culture media known in the art that maintains the pluripotency of the ESCs, with or without feeder cells, such as murine embryonic feeder cells (MEF) cells or human feeder cells, such as human dermal fibroblasts (HDF). The MEF cells or human feeder cells may be mitotically inactivated, for example, by exposure to mitomycin C, gamma irradiation, or by any other known methods, prior to seeding ESCs in co-culture, and thus the MEFs do not propagate in culture. Additionally, ESC cell cultures may be examined microscopically and colonies containing non ESC cell morphology may be picked and discarded, e.g., using a stem cell cutting tool, by laser ablation, or other means. Typically, after the point of harvest of the ESCs for seeding for embryoid body formation no additional MEF cells or human feeder cells are used.

Alternatively, hES cells may be cultured under feeder-free conditions on a solid surface such as an extracellular matrix (e.g., Matrigel®, laminin, or iMatrix-511 or any other extracellular matrix disclosed herein or known in the art) by any method known in the art, e.g., Klimanskaya et al., Lancet 365:1636-1641 (2005). Accordingly, the hES cells used in the methods described herein may be cultured on feeder-free cultures.

“Embryo-derived cells” (EDC), as used herein, refers broadly to pluripotent morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

“Induced pluripotent stem cells” or “iPSCs” or “iPS cells” as used herein refer to pluripotent stem cells generated by reprogramming a somatic cell. iPSCs may be generated by expressing or inducing expression of a combination of factors (“reprogramming factors”). iPS cells may be generated using fetal, postnatal, newborn, juvenile, or adult somatic cells. iPS cells may be obtained from a cell bank. Alternatively, iPS cells may be newly generated (by processes known in the art) prior to commencing differentiation to vascular progenitor cells (VPCs) or another cell type. The making of iPS cells may be an initial step in the production of differentiated cells. iPS cells may be specifically generated using material from a particular patient or matched donor with the goal of generating tissue-matched VPCs. iPS cells can be produced from cells that are not substantially immunogenic in an intended recipient, e.g., produced from autologous cells or from cells histocompatible to an intended recipient. As further discussed above (see “pluripotent cells”), pluripotent cells including iPS cells may be genetically modified or otherwise modified. An exemplary human iPSC cell line that may be used is GMP1 cells.

As a further example, induced pluripotent stem cells may be generated by reprogramming a somatic or other cell by contacting the cell with one or more reprogramming factors. For example, the reprogramming factor(s) may be expressed by the cell, e.g., from an exogenous nucleic acid added to the cell, or from an endogenous gene in response to a factor such as a small molecule, microRNA, or the like that promotes or induces expression of that gene (see Suh and Blelloch, Development 138, 1653-1661 (2011); Miyoshi et al., Cell Stem Cell (2011), doi:10.1016/j.stem.2011.05.001; Sancho-Martinez et al., Journal of Molecular Cell Biology (2011) 1-3; Anokye-Danso et al., Cell Stem Cell 8, 376-388, Apr. 8, 2011; Orkin and Hochedlinger, Cell 145, 835-850, Jun. 10, 2011, or Warren et al., Scientific Reports, 10.1038/srep00657, Sep. 14, 2012, each of which is incorporated by reference herein in its entirety). Reprogramming factors may be provided from an exogenous source, e.g., by being added to the culture media, and may be introduced into cells by methods known in the art such as through coupling to cell entry peptides, protein or nucleic acid transfection agents, lipofection, electroporation, biolistic particle delivery system (gene gun), microinjection, and the like. In certain embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct-4, Sox2, Nanog, and Lin28. In other embodiments, somatic cells are reprogrammed by expressing at least 2 reprogramming factors, at least three reprogramming factors, or four reprogramming factors. In another embodiment, somatic cells are reprogrammed by expressing Oct4, Sox2, MYC, Klf4, Nanog, and Lin28. In other embodiments, additional reprogramming factors are identified and used alone or in combination with one or more known reprogramming factors to reprogram a somatic cell to a pluripotent stem cell. iPS cells typically can be identified by expression of the same markers as embryonic stem cells, though a particular iPS cell line may vary in its expression profile.

The induced pluripotent stem cell may be produced by expressing or inducing the expression of one or more reprogramming factors in a somatic cell. In an embodiment, the somatic cell is a fibroblast, such as a dermal fibroblast, synovial fibroblast, or lung fibroblast, or a non-fibroblastic somatic cell. In an embodiment, the somatic cell is reprogrammed by expressing at least 1, 2, 3, 4, 5 reprogramming factors as described above. In another embodiment, expression of the reprogramming factors may be induced by contacting the somatic cells with at least one agent, such as a small organic molecule agents, that induce expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule.) For example, reprogramming factors may be expressed in the somatic cell by infection using a viral vector, such as a retroviral vector or a lentiviral vector. Also, reprogramming factors may be expressed in the somatic cell using a non-integrative vector, such as an episomal plasmid or mRNA. See, e.g., Yu et al., Science. 2009 May 8; 324(5928):797-801, which is hereby incorporated by reference in its entirety. When reprogramming factors are expressed using non-integrative vectors, the factors may be expressed in the cells using electroporation, transfection, or transformation of the somatic cells with the vectors.

Once the reprogramming factors are expressed in the cells, the cells may be cultured by any method known in the art. Over time, cells with ES characteristics appear in the culture dish. The cells may be chosen and subcultured based on, for example, ES morphology, or based on expression of a selectable or detectable marker. The cells may be cultured to produce a culture of cells that resemble ES cells—these are putative iPS cells. iPS cells typically can be identified by expression of the same markers as other embryonic stem cells, though a particular iPS cell line may vary in its expression profile. Exemplary iPS cells may express Oct-4, alkaline phosphatase, SSEA 3 surface antigen, SSEA 4 surface antigen, TRA 1 60, and/or TRA 1 81.

To confirm the pluripotency of the iPS cells, the cells may be tested in one or more assays of pluripotency. For example, the cells may be tested for expression of ES cell markers; the cells may be evaluated for ability to produce teratomas when transplanted into SCID mice; the cells may be evaluated for ability to differentiate to produce cell types of all three germ layers. Once a pluripotent iPS cell is obtained it may be used to produce mesoderm cells and vascular progenitor cells, e.g., mesoderm-derived vascular progenitor cells.

“Mesoderm” as used herein refers to one of the three primary germ layers in the very early embryo of all belaterian animals. The mesoderm forms mesenchyme, mesothelium, non-epithelial blood cells and coelomocytes. Early mesoderm commitment arises from an epithelial to mesenchymal transition following which the specified mesodermal lineage cells migrate inward as gastrulation proceeds. Cells of the mesodermal lineage are fated to form the vascular and lymphatic systems, including hemangioblasts and multipotent mesenchymal stem cells capable of differentiating into multiple specified cell types. Mesoderm gives rise to vasculogenesis through the formation of extraembryonic mesoderm and then embryonic splanchnic mesoderm. Growth factors such as vascular endothelial growth factor (VEGF) and placental growth factor (PIGF or PGF) stimulate the growth and development of new blood vessels. In one embodiment, cells of the mesodermal lineage are fated to be vascular precursor cells or vascular progenitor cells. In one embodiment, pluripotent stem cells, e.g., hESCs or iPSCs, e.g., hiPSCs can be differentiated into mesodermal lineaged cells, e.g., mesoderm precursor cells. Thus, the term “mesoderm” also includes mesoderm lineaged cells derived from pluripotent stem cells, regardless of the maturity of the cells, and thus the term encompasses mesoderm cells of various levels of maturity, including mesoderm precursor cells.

Exemplary mesodermal markers include, but are not limited to, CD309/KDR, CD56/NCAM1, APLNR/APJ, GARP, CD13, N-Cadherin, Activin A, Activin AB, Activin AC, Activin B, Activin C, BMP and other Activin receptor activators, BMP and other Activin receptor inhibitors, BMP-2, BMP-2/BMP-4, BMP-2/BMP-6 Heterodimer, BMP-2/BMP-7 Heterodimer, BMP-2a, BMP-4, BMP-6, BMP-7, Cryptic, FABP4/A-FABP, FGF-5, GDF-1, GDF-3, INHBA, INHBB, Nodal, TGF-beta, TGF-beta 1, TGF-beta 1, 2, 3, TGF-beta 1.2, TGF-beta 1/1.2, TGF-beta 2, TGF-beta 2/1.2, TGF-beta 3, TGF-beta Receptor Inhibitors, Wnt-3a, Wnt-8a, MESDC2, Nicalin, Brachyury, EOMES, FoxC1, FoxF1, Goosecoid, HAND1, MIXL1, Slug, Snail, TBX6, Twist-1, and Twist-2. In one embodiment, the mesoderm cells are mesoderm precursor cells, which are positive for one or more markers selected from CD309/KDR, CD56/NCAM1, APLNR/APJ, GARP, and CD13.

“Vasculogenesis” as used herein, refers to the formation of new blood vessels. Vasculogenesis includes the formation of endothelium derived from the mesoderm. “Angiogenesis” as used herein, refers to the formation of blood vessels from pre-existing vessels. See, e.g., Developmental Biology by Gilbert, Scott F. Sunderland (Mass.): Sinauer Associates, Inc.; c2000, and Molecular Biology of the Cell 4th ed. Alberts, Bruce; Johnson, Alexander; Lewis, Julian; Raff, Martin; Roberts, Keith; Walter, Peter New York and London: Garland Science; c2002.

“Vascular progenitor cells” (VPCs) as used herein, refers to cells that have the capacity to differentiate into endothelial cells, smooth muscle cells, and pericytes, among other hemato-vascular cell lineages. In one embodiment, a vascular progenitor cell is a mesoderm-derived vascular progenitor cell (meso-VPC).

“Mesoderm-derived vascular progenitor cells” (meso-VPCs) as used herein, refers to VPCs that are generated from mesoderm cells derived by the in vitro differentiation of pluripotent stem cells, e.g., ESCs or iPSCs. Meso-VPCs may be identified by the expression of one or more cell-surface markers as further described herein. In one embodiment, mesoderm-derived vascular progenitor cells are generated from the in vitro differentiation of pluripotent stem cells, e.g., ESCs or iPSCs into mesoderm cells which, in turn, are differentiated into meso-VPCs.

Meso-VPCs may be derived in vitro from both mouse PSCs and human PSCs. Meso-VPCs are capable of differentiating into hematopoietic and endothelial cell lineages, and may be capable of also becoming smooth muscle cells. The population of meso-VPCs of the current invention may be positive for at least one marker such as CD31/PECAM1, CD309/KDR, CD43, CD144, CD34, CD184/CXCR4, CD146, and PDGFRb. In one embodiment, the population of meso-VPC is positive for 1, 2, 3, 4, 5, 6, 7, or 8 of the above-identified markers. In one embodiment, the population of meso-VPC is positive for CD146, CD31/PECAM1, and CD309/KDR. In another embodiment, the population of meso-VPCs express CD31/PECAM1, CD309/KDR, CD146, and (i) at least one of CD144, CD34, CD184/CXCR4, CD43, or PDGFRb, (ii) CD34, CD184/CXCR4, and PDGFRb; (iii) CD184/CXCR4; (iv) PDGFRb; (v) CD144 and CD184/CXCR4; (vi) CD184/CXCR4 and CD43; or (vii) CC184/CXCFR4. In an embodiment, the population of meso-VPCs expresses at least 1, at least 2, at least 3, at least 4, at least 5, at least 6, at least 7, at least 8, at least 9, at least 10, at least 11, at least 12, at least 13, at least 14, or at least 15 miRNA markers selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p. In one embodiment, the population of meso-VPCs expresses hsa-miR-3917, hsa-miR-450a-2-3p, and hsa-miR-542-5p. In an embodiment, the population of meso-VPCs are considered expressing a certain marker if at least about 20% of the meso-VPCs in a composition express the marker. In one embodiment, the meso-VPC of the present invention is positive for at least one, at least two, at least three, or at least four miRNA markers selected from the group consisting of mir126, mir125a-5p, mir24, and mir483-5p. In one embodiment, the miRNA marker is mir483-5p. In one embodiment, the population of meso-VPCs comprises at least one meso-VPC that is positive for at least one, at least two, at least three, or at least four miRNA markers selected from the group consisting of mir126, mir125a-5p, mir24, and mir483-5p. In one embodiment, the miRNA marker is mir483-5p. In an embodiment, the population of meso-VPCs express CD31 and KDR at a higher level than the population of HE cells. In another embodiment, the population of meso-VPCs expresses CD146 at a lower level than the population of HE cells. In yet another embodiment, the population of meso-VPCs expresses CD184/CXCR4 at a lower level than the population of HE cells.

In any of the embodiments, the population of meso-VPCs exhibits limited or no detection of one, two, or three of CXCR7, CD45, and NG2. In any of the embodiments, the population of meso-VPCs exhibits limited or no detection of all of CXCR7, CD45, and NG2. In any of the embodiment, the population of meso-VPCs exhibits limited or no detection of at least 1, at least 2, at least 3, at least 4, at least 5, at least 6, at least 7, or at least 8 of CD144, CD34, CD184/CXCR4, CXCR7, CD43, CD45, PDGFRb, or NG2. In an embodiment, the population of meso-VPCs exhibits limited or no expression of at least 1, at least 2, at least 3, at least 4, at least 5, at least 6, or at least 7 miRNA markers selected from hsa-let-7e-3p, hsa-miR-99a-3p, hsa-miR-133a-5p, hsa-miR-11399, hsa-miR-196b-3p, hsa-miR-5690, and hsa-miR-7151-3p. In an embodiment, the population of meso-VPCs is considered exhibiting limited or no detection of a marker if less than about 20% of the meso-VPCs in a composition express the marker. In an embodiment, the meso-VPC of the present invention exhibits limited or no expression for at least 1, at least 2, at least 3, at least 4, at least 5, at least 6, at least 7, at least 8, or at least 9 miRNA markers selected from the group consisting of mir367, mir302a, mir302b, mir302c, mirLet7-e, mir223, mir99a, mir142-3p, and mir133a. In an embodiment of the present invention, the population of meso-VPCs comprises at least one meso-VPC that exhibits limited or no expression for at least 1, at least 2, at least 3, at least 4, at least 5, at least 6, at least 7, at least 8, or at least 9 miRNA markers selected from the group consisting of mir367, mir302a, mir302b, mir302c, mirLet7-e, mir223, mir99a, mir142-3p, and mir133a.

“Vasculonoid” as used herein, refers to a colony-like aggregate of cells, e.g., mesoderm-derived vascular progenitor cells (meso-VPCs) formed during, e.g., cell culture. In one embodiment, a vasculonoid is formed by meso-VPCs produced using the 3D-Vasculonoid differentiation platform.

“Therapy,” “therapeutic,” “treating,” “treat” or “treatment”, as used herein, refers broadly to treating a disease, arresting or reducing the development of the disease or its clinical symptoms, and/or relieving the disease, causing regression of the disease or its clinical symptoms. “Therapy”, “therapeutic,” “treating,” “treat” or “treatment” encompasses prophylaxis, prevention, treatment, cure, remedy, reduction, alleviation, and/or providing relief from a disease, signs, and/or symptoms of a disease. “Therapy”, “therapeutic,” “treating,” “treat” or “treatment” encompasses an alleviation of signs and/or symptoms in patients with ongoing disease signs and/or symptoms. “Therapy”, “therapeutic,” “treating,” “treat” or “treatment” also encompasses “prophylaxis” and “prevention”. Prophylaxis includes preventing disease occurring subsequent to treatment of a disease in a patient or reducing the incidence or severity of the disease in a patient. The term “reduced”, for purpose of therapy, “therapeutic,” “treating,” “treat” or “treatment” refers broadly to the clinical significant reduction in signs and/or symptoms. “Therapy”, “therapeutic,” “treating,” “treat” or “treatment” includes treating relapses or recurrent signs and/or symptoms. “Therapy”, “therapeutic,” “treating,” “treat” or “treatment” encompasses but is not limited to precluding the appearance of signs and/or symptoms anytime as well as reducing existing signs and/or symptoms and eliminating existing signs and/or symptoms. “Therapy”, “therapeutic,” “treating,” “treat” or “treatment” includes treating chronic disease (“maintenance”) and acute disease. For example, treatment includes treating or preventing relapses or the recurrence of signs and/or symptoms. In one embodiment, treatment includes clinical significant reduction in signs and/or symptoms of a vascular disease, such as critical limb ischemia.

“Normalizing a pathology”, as used herein, refers to reverting the abnormal structure and/or function resulting from a disease to a more normal state. Normalization suggests that by correcting the abnormalities in structure and/or function of a tissue, organ, or cell type resulting from a disease, the progression of the pathology can be controlled and improved. For example, following treatment with the meso-VPCs of the present invention the abnormalities of the limb as a result of a vascular disease, e.g., critical limb ischemia, may be improved, corrected, and/or reversed.

“Vascular diseases” as used herein, refer to any abnormal condition of the blood vessels (arteries and veins). Vascular diseases outside the heart can present themselves anywhere. The most common vascular diseases are stroke, peripheral artery disease (PAD), abdominal aortic aneurysm (AAA), carotid artery disease (CAD), arteriovenous malformation (AVM), critical limb ischemia (CLI), pulmonary embolism (blood clots), deep vein thrombosis (DVT), chronic venous insufficiency (CVI), and varicose veins. In one embodiment, the vascular disease is a peripheral artery disease (PAD). In one embodiment, the vascular disease is an ischemic disease, such as critical limb ischemia (CLI). In one embodiment, the vascular disease is atherosclerosis, peripheral artery disease (PAD), carotid artery disease, venous disease, blood clots, aortic aneurysm, fibromuscular dysplasia, lymphedema, or vascular injury. In one embodiment, the vascular disease is a periphery artery disease such as critical limb ischemia (CLI), intestinal ischemic syndrome, renal artery disease, popliteal entrapment syndrome, Raynaud's phenomenon, or Buerger's disease.

### II. In Vitro Generation of Mesoderm-Derived Vascular Progenitor Cells (Meso-VPCs)

The current invention provides methods of producing a mesoderm-derived vascular progenitor cell (meso-VPC) from a mesoderm cell derived from a pluripotent stem cell. The methods include the steps of culturing a pluripotent stem cell in a medium containing one or more mesoderm inducing growth factors to produce a mesoderm cell, and culturing the mesoderm cell on an appropriate surface, in a medium containing one or more factors that direct the differentiation of the mesoderm cell into a mesoderm-derived vascular progenitor cell (meso-VPC). In some embodiments, the methods further include dissociating a plurality of meso-VPCs into single cells.

Pluripotent stems cells used in the current invention can be obtained and cultured by any of the methods presented above. In one embodiment, pluripotent stem cells, e.g., human embryonic stem cells (hESCs) or human induced pluripotent stem cells (hiPSCs), are cultured in feeder-free (FF) conditions and plated on an extracellular matrix. In one embodiment, the pluripotent stem cells are cultured in feeder culture conditions and plated on an extracellular matrix.

In some embodiments, the extracellular matrix is selected from the group consisting of laminin, fibronectin, vitronectin, proteoglycan, entactin, collagen, collagen I, collagen IV, heparan sulfate, a soluble preparation from Engelbreth-Holm-Swarm (EHS) mouse sarcoma cells, Matrigel® (Corning), gelatin, and a human basement membrane extract. In one embodiment, the extracellular matrix may be derived from any mammalian, including human, origin. In one embodiment, the extracellular matrix surface for culturing the pluripotent stem cells is a Matrigel-coated surface.

In some embodiments, the pluripotent stem cells are cultured in a medium suitable for supporting pluripotency and any such medium are known in the art. In some embodiments, the medium that supports pluripotency is Nutristem™. In some embodiments, the medium that supports pluripotency is TeSR™. In some embodiments, the medium that supports pluripotency is StemFit™. In other embodiments, the medium that supports pluripotency is Knockout™ DMEM (Gibco), which may be supplemented with Knockout™ Serum Replacement (Gibco), LIF, bFGF, or any other factors. Each of these exemplary media is known in the art and commercially available. In further embodiments, the medium that supports pluripotency may be supplemented with bFGF or any other factors. In an embodiment, bFGF may be supplemented at a low concentration (eg. 4 ng/mL). In another embodiment, bFGF may be supplemented at a higher concentration (eg. 100 ng/mL). In an embodiment, the medium is serum-free. In another embodiment, the medium comprises serum.

The pluripotent stem cells can be cultured, passaged or harvested in any suitable containers known in the art. Exemplary tissue culture containers include 15 cm tissue culture plates, 10 cm tissue culture plates, 3 cm tissue culture plates, 6-well tissue culture plates, 12-well tissue culture plates, 24-well tissue culture plates, 48-well tissue culture plates, 96-well, tissue culture plates, T-25 tissue culture flasks, T-75 tissue culture flasks. In one embodiment, the pluripotent stem cells are cultured in a 6-well tissue culture plate.

In some embodiments, medium change is performed after about 1, 2, 3, 4, 5, or 6 days of culture to maintain the optimal condition of the pluripotent stem cells. For medium change, the same culture media as the starting condition may be used, or the medium may be adjusted according to culturing needs. In some embodiments, the pluripotent stem cells are split and passaged after about 1, 2, 3, 4, 5, 6, 7, 8, or 9 days, or when the cell culture reaches about 60-90% confluency. For cell passage, the same culture media as the starting condition may be used, or the medium may be adjusted according to culturing needs. The cells may be split and passaged at a 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 ratio of dilution. In one embodiment, the pluripotent stem cells are passaged at a 1:3 ratio of dilution.

In some embodiments, the pluripotent stem cells may be cultured under a normoxia condition of about 5% CO₂ and about 20% O₂, or other known conditions suitable for the growth of pluripotent stem cells.

In some embodiments, the pluripotent stem cells are cultured, passaged or harvested in culture medium under feeder-free conditions wherein no feeder layer of cells are contained in the culture. In some embodiments, the pluripotent stem cells are cultured, passaged or harvested in culture medium under feeder culture conditions wherein a layer of feeder cells such as human dermal fibroblasts (HDFs), or other cell types known to one of ordinary skill in the art are contained in the culture.

To produce mesoderm cells by in vitro differentiation of pluripotent stem cells, pluripotent stem cells, e.g., hESCs or hiPSCs, are cultured on a suitable surface, e.g., an extracellular matrix surface. In some embodiments, the extracellular matrix is selected from the group consisting of laminin, fibronectin, vitronectin, proteoglycan, entactin, collagen, collagen I, collagen IV, heparan sulfate, a soluble preparation from Engelbreth-Holm-Swarm (EHS) mouse sarcoma cells, Matrigel, gelatin, and a human basement membrane extract. In one embodiment, the extracellular matrix may be derived from any mammalian, including human, origin. In one embodiment, the extracellular matrix surface for in vitro differentiation of pluripotent stem cells into mesoderm cells is a Matrigel-coated surface.

In one embodiment, pluripotent stem cells are plated and cultured for about 1 hour to about 24 hours in the culture media to let the cells settle before inducing differentiation. To induce the differentiation of the pluripotent stem cells into mesoderm cells, the pluripotent stem cells are cultured in a culture medium on a suitable surface, e.g., an extracellular matrix surface as described above.

The culture media for inducing differentiation of the pluripotent stem cells into mesoderm cells may be any medium that supports differentiation and may be a culture media known in the art. In some embodiments, the culture media may be any medium that supports hemato-vascular culture and/or expansion, and includes, but is not limited to, Stemline® II (Sigma), StemSpan™ SFEMII (StemCell Technologies), StemSpan™ AFC (StemCell Technologies), Minimal Essential Media (MEM) (Gibco), and αMEM. In an embodiment, the culture medium is serum-free. In another embodiment, the culture medium comprises serum. The culture media may further comprise one or more mesoderm inducing growth factors such as Activin-A, vascular endothelial growth factor (VEGF), fibroblast growth factor (FGF), and bone morphogenetic protein 4 (BMP4). In one embodiment, the VEGF used in the method is VEGF165. In one embodiment, the FGF used in the method is basic FGF (bFGF). In one embodiment, the pluripotent stem cells are cultured in a culture media comprising Activin-A, VEGF165, bFGF and BMP4. In one embodiment, the culturing duration is about 1, 2, 3, 4, 5, 6, or 7 days. In one embodiment, the culturing duration is about 4 days. In one embodiment, the culture media is changed after about 24 hours of culturing and is replaced by a culture media without Activin-A.

The VEGF, e.g., VEGF165, can be used at a concentration of about 1 ng/mL to about 100 ng/mL, or more preferably, about 5 ng/mL to about 20 ng/mL. In one embodiment, the VEGF is used at a concentration of about 1 ng/mL, about 2 ng/mL, about 3 ng/mL, about 4 ng/mL, about 5 ng/mL, about 10 ng/mL, about 15 ng/mL, or about 20 ng/mL. The Activin-A can be used at a concentration of about 1 ng/mL to about 100 ng/mL, or more preferably about 5 ng/mL to about 20 ng/mL. In one embodiment, the Activin-A is used at a concentration of about 1 ng/mL, about 2 ng/mL, about 3 ng/mL, about 4 ng/mL, about 5 ng/mL, about 10 ng/mL, about 15 ng/mL, or about 20 ng/mL. The FGF, e.g., bFGF, can be used at a concentration of about 1 ng/mL to about 100 ng/mL, or more preferably about 5 ng/mL to about 20 ng/mL. In one embodiment, the FGF is used at a concentration of about 1 ng/mL, about 2 ng/mL, about 3 ng/mL, about 4 ng/mL, about 5 ng/mL, about 10 ng/mL, about 15 ng/mL, or about 20 ng/mL. The BMP4 can be used at a concentration of about 1 ng/mL to about 100 ng/mL, or more preferably about 5 ng/mL to about 35 ng/mL. In one embodiment, the BMP4 is used at a concentration of about 1 ng/mL, about 2 ng/mL, about 3 ng/mL, about 4 ng/mL, about 5 ng/mL, about 10 ng/mL, about 15 ng/mL, about 20 ng/mL, about 25 ng/mL, about 30 ng/mL, or about 35 ng/mL. In one embodiment, the VEGF is used at a concentration of 10 ng/mL, the Activin-A is used at a concentration of 10 ng/mL, the FGF is used at a concentration of 10 ng/mL, and the BMP4 is used at a concentration of 25 ng/mL.

The differentiation of pluripotent stem cells into mesoderm cells may be performed under a normoxia condition of about 5% CO₂ and about 20% O₂, or other known conditions suitable for the differentiation of pluripotent stem cells.

The differentiation of pluripotent stem cells into mesoderm cells may be conducted in any suitable containers known in the art. Exemplary tissue culture containers include, but are not limited to, 15 cm tissue culture plates, 10 cm tissue culture plates, 3 cm tissue culture plates, 6-well tissue culture plates, 12-well tissue culture plates, 24-well tissue culture plates, 48-well tissue culture plates, 96-well, tissue culture plates, T-25 tissue culture flasks, and T-75 tissue culture flasks. In one embodiment, the differentiation of pluripotent stem cells in to mesoderm cells is conducted in a 10 cm tissue culture plate.

The mesoderm cells may be further dissociated into single cells for further uses. In one embodiment, the mesoderm cells produced by in vitro differentiation of pluripotent stem cells are dissociated by enzymatic treatment into single cells.

In one embodiment, the mesoderm cells express at least 1, at least 2, at least 3, at least 4, or at least 5 markers selected from the group comprising CD309/KDR, CD56/NCAM1, APLNR/APJ, GARP, and CD13.

The mesoderm cells may also express one or more other mesodermal markers selected from the group consisting of N-Cadherin, Activin A, Activin AB, Activin AC, Activin B, Activin C, BMP and other Activin receptor activators, BMP and other Activin receptor inhibitors, BMP-2, BMP-2/BMP-4, BMP-2/BMP-6 Heterodimer, BMP-2/BMP-7 Heterodimer, BMP-2a, BMP-4, BMP-6, BMP-7, Cryptic, FABP4/A-FABP, FGF-5, GDF-1, GDF-3, INHBA, INHBB, Nodal, TGF-beta, TGF-beta 1, TGF-beta 1, 2, 3, TGF-beta 1.2, TGF-beta 1/1.2, TGF-beta 2, TGF-beta 2/1.2, TGF-beta 3, TGF-beta Receptor Inhibitors, Wnt-3a, Wnt-8a, MESDC2, Nicalin, Brachyury, EOMES, FoxC1, FoxF1, Goosecoid, HAND1, MIXL1, Slug, Snail, TBX6, Twist-1, and Twist-2.

The mesoderm cells produced by the methods of the invention are further differentiated into mesoderm-derived vascular progenitor cells (meso-VPCs) using one of the two platforms disclosed herein: the 3D-Vasculonoid differentiation platform or the 2D differentiation platform.

The 3D-Vasculonoid differentiation platform provides methods for in vitro differentiation of mesoderm cells produced from pluripotent stem cells, e.g., hESCs or hiPSCs, into meso-VPCs.

The methods of the 3D-Vasculonoid differentiation platform are performed by culturing the mesoderm cells in a culture medium under non-adherent or low adherent conditions, e.g., on an ultra-low attachment surface or suspension culture, wherein the culture media may be any culture media that supports differentiation and may be known in the art. In some embodiments, the culture media may be any medium that supports hemato-vascular culture and/or expansion, and includes, but is not limited to, Stemline® II (Sigma), StemSpan™ SFEMII (StemCell Technologies), StemSpan™ AFC (StemCell Technologies), Minimal Essential Media (MEM) (Gibco), and αMEM. In an embodiment, the culture medium is serum-free. In another embodiment, the culture medium comprises serum. The culture media may further comprise one or more factors that induce the differentiation of mesoderm cells into meso-VPCs, e.g., vascular endothelial growth factor (VEGF), fibroblast growth factor (FGF), bone morphogenetic protein 4 (BMP4), a small molecule inhibitor of transforming growth factor-beta (TGF-β) type I receptor, and Forskolin. In one embodiment, the VEGF used in the method is VEGF165. In one embodiment, the FGF used in the method is basic FGF (bFGF). In one embodiment, the small molecule inhibitor of transforming growth factor-beta (TGF-β) type I receptor is SB431542. In one embodiment, the pluripotent stem cells are cultured in a culture media comprising VEGF165, bFGF, BMP4, and SB431542. In one embodiment, the pluripotent stem cells are cultured in a culture media comprising VEGF165, bFGF, BMP4, SB431542, and Forksolin. In one embodiment, the culture duration is about 1, 2, 3, 4, 5, 6, 7, 8, 9, or 10 days. In one embodiment, the culture duration is about 5 days. In one embodiment, the culture media is changed after about 2 days and after about 4 days of the start of the differentiation.

The VEGF, e.g., VEGF165, can be used at a concentration of about 1 ng/mL to about 100 ng/mL, or more preferably, about 10 ng/mL to about 100 ng/mL. In one embodiment, the VEGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The FGF, e.g., bFGF, can be used at a concentration of about 1 ng/mL to about 100 ng/mL, or more preferably, about 10 ng/mL to about 100 ng/mL. In one embodiment, the FGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The BMP4 can be used at a concentration of about 1 ng/mL to about 100 ng/mL, or more preferably, about 10 ng/mL to about 100 ng/mL. In one embodiment, the BMP4 is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The small molecule inhibitor of transforming growth factor-beta (TGF-β) type I receptor, e.g., SB431542, can be used at a concentration of about 0.1 μM to about 100 or more preferably about 1 μM to about 100 μM. In one embodiment, the small molecule inhibitor of transforming growth factor-beta (TGF-β) type I receptor is used at a concentration of about 0.1 μM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 60 μM, 65 μM, 70 μM, 75 μM, 80 μM, 85 μM, 90 μM, 95 μM, 95 μM, or 100 μM. The Forskolin can be used at a concentration of about 0.1 μM to about 10 μM. In one embodiment, the Forskolin is used at a concentration of about 0.1 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM.

In one embodiment, the VEGF is used at a concentration of about 50 ng/mL, the FGF is used at a concentration of about 50 ng/mL, the BMP4 is used at a concentration of about 25 ng/mL, the small molecule inhibitor is used at a concentration of about 10 and the Forskolin is used at a concentration of about 2 μM.

The differentiation of mesoderm cells into meso-VPCs using the 3D-Vasculonoid differentiation platform may be performed under a normoxia condition of about 5% CO₂ and about 20% O₂, or other known conditions suitable for the differentiation of pluripotent stem cells.

The differentiation of mesoderm cells into meso-VPCs using the 3D-Vasculonoid differentiation platform may be conducted in any suitable containers known in the art. Exemplary tissue culture containers include, but are not limited to, 15 cm tissue culture plates, 10 cm tissue culture plates, 3 cm tissue culture plates, 6-well tissue culture plates, 12-well tissue culture plates, 24-well tissue culture plates, 48-well tissue culture plates, 96-well, tissue culture plates, T-25 tissue culture flasks, and T-75 tissue culture flasks. In one embodiment, the differentiation of mesoderm cells into meso-VPCs using the 3D-Vasculonoid differentiation platform is conducted in a 10 cm tissue culture plate.

The differentiation of mesoderm cells into meso-VPCs using the 3D-Vasculonoid differentiation platform may be conducted in non-adherent or low adherent conditions under which the cells minimally adhere to the culture vessel. In one embodiment, the differentiation of mesoderm cells into meso-VPCs using the 3D-Vasculonoid differentiation platform is conducted on an ultra-low attachment surface or suspension culture.

In some embodiments, the meso-VPCs produced by the 3D-Vasculonoid differentiation platform form vasculonoids. Vasculonoids, as used herein, refers to cell aggregates, for example, colony-like aggregates that are formed by vascular cell lineages, e.g., meso-VPCs. The morphology of the vasculonoids may vary depending on methods used to produce the vascular cells. The current invention further provides methods of dissociating the plurality of cells in the vasculonoids to obtain single cells. In one embodiment, the meso-VPCs produced by the 3D-Vasculonoid differentiation platform may be further dissociated into single cells. In one embodiment, the plurality of meso-VPCs in the vasculonoid are dissociated into single cells by enzymatic treatment.

The 2D differentiation platform provides methods for in vitro differentiation of mesoderm cells produced from pluripotent stem cells, e.g., hESCs or hiPSCs, into meso-VPCs.

The methods of the 2D differentiation platform are performed by culturing the mesoderm cells in a culture medium on a suitable surface, e.g., an extracellular matrix surface. In some embodiments, the extracellular matrix is selected from the group consisting of laminin, fibronectin, vitronectin, proteoglycan, entactin, collagen, collagen I, collagen IV, heparan sulfate, a soluble preparation from Engelbreth-Holm-Swarm (EHS) mouse sarcoma cells, Matrigel, gelatin, and a human basement membrane extract. In one embodiment, the extracellular matrix may be derived from any mammalian, including human, origin. In one embodiment, the extracellular matrix surface for in vitro differentiation of mesoderm cells is a collagen IV-coated surface.

The culture media may be may be any medium that supports differentiation of the mesoderm cells and may be a culture media known in the art. In some embodiments, the culture media may be any medium that supports hemato-vascular culture and/or expansion, and includes, but is not limited to, Stemline® II (Sigma), StemSpan™ SFEMII (StemCell Technologies), StemSpan™ AFC (StemCell Technologies), Minimal Essential Media (MEM) (Gibco), and αMEM. In an embodiment, the culture medium is serum-free. In another embodiment, the culture medium comprises serum. The culture media may further comprise one or more factors that induce the differentiation of mesoderm cells into meso-VPCs. The factors are selected from vascular endothelial growth factor (VEGF), fibroblast growth factor (FGF), bone morphogenetic protein 4 (BMP4), a small molecule inhibitor of transforming growth factor-beta (TGF-β) type I receptor, and Forskolin. In one embodiment, the VEGF used in the method is VEGF165. In one embodiment, the FGF used in the method is basic FGF (bFGF). In one embodiment, the small molecule inhibitor of transforming growth factor-beta (TGF-β) type I receptor is SB431542.

In an embodiment, the methods of the 2D differentiation platform for differentiating mesoderm cells to obtain meso-VPCs comprise two steps. The mesoderm cells are first differentiated in a culture medium that supports differentiation and may be a culture medium known in the art. In some embodiments, the culture medium may be any medium that supports hemato-vascular culture and/or expansion, and includes, but is not limited to, Stemline® II (Sigma), StemSpan™ SFEMII (StemCell Technologies), StemSpan™ AFC (StemCell Technologies), Minimal Essential Media (MEM) (Gibco), and αMEM. In an embodiment, the culture medium is serum-free. In another embodiment, the culture medium comprises serum. The culture medium may further comprise one or more factors selected from vascular endothelial growth factor (VEGF), fibroblast growth factor (FGF), bone morphogenetic protein 4 (BMP4), and Forskolin. In one embodiment, the culture medium comprises VEGF165, bFGF, and BMP4. In one embodiment, the culture medium comprises VEGF165, bFGF, BMP4, and Forskolin. The culturing in this step is performed for about 12 hours to about 2 days. In one embodiment, the first step of the 2D differentiation platform to differentiate the mesoderm cells into meso-VPCs is performed for about 1 day.

The VEGF, e.g., VEGF165, can be used at a concentration of about 1 ng/mL to about 100 ng/mL, or more preferably, 10 ng/mL to about 100 ng/mL. In one embodiment, the VEGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The FGF, e.g., bFGF, can be used at a concentration of about 1 ng/mL to about 100 ng/mL, or more preferably, about 10 ng/mL to about 100 ng/mL. In one embodiment, the FGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The BMP4 can be used at a concentration of about 1 ng/mL to about 100 ng/mL, or more preferably, about 10 ng/mL to about 100 ng/mL. In one embodiment, the BMP4 is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The Forskolin can be used at a concentration of about 0.1 μM to about 10 μM. In one embodiment, the Forskolin is used at a concentration of about 0.1 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM.

In one embodiment, the VEGF is used at a concentration of about 50 ng/mL, the FGF is used at a concentration of about 50 ng/mL, the BMP4 is used at a concentration of about 25 ng/mL, and the Forskolin is used at a concentration of about 2 μM.

The first step of differentiation of mesoderm cells into meso-VPCs using the 2D differentiation platform may be performed under a normoxia condition of about 5% CO₂ and about 20% O₂, or other known conditions suitable for the differentiation of mesoderm cells.

The second step of the 2D differentiation platform further differentiates the cells obtained in the first step into meso-VPCs, in a culture medium that supports differentiation. In some embodiments, the culture medium may be any medium that supports hemato-vascular culture and/or expansion, and includes, but is not limited to, Stemline® II (Sigma), StemSpan™ SFEMII (StemCell Technologies), StemSpan™ AFC (StemCell Technologies), Minimal Essential Media (MEM) (Gibco), and αMEM. In an embodiment, the culture medium is serum-free. In another embodiment, the culture medium comprises serum. The culture medium may further comprise one or more factors, such as vascular endothelial growth factor (VEGF), fibroblast growth factor (FGF), bone morphogenetic protein 4 (BMP4), a small molecule inhibitor of transforming growth factor-beta (TGF-β) type I receptor, and/or Forskolin. In one embodiment, the culture medium comprises VEGF165, bFGF, BMP4, and SB431542. In one embodiment, the culture medium comprises VEGF165, bFGF, BMP4, SB431542, and Forskolin. The culturing in this step is performed for about 1, 2, 3, 4, 5, 6, 7, 8, 9, or 10 days. In one embodiment, the second step of the 2D differentiation platform to differentiate the mesoderm cells into meso-VPCs is performed for about 6 days. In one embodiment, the culture media is changed after about 2 days and after about 4 days of the start of the second step of the 2D differentiation platform.

The VEGF, e.g., VEGF165, can be used at a concentration of about 1 ng/mL to about 100 ng/mL, or more preferably, 10 ng/mL to about 100 ng/mL. In one embodiment, the VEGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The FGF, e.g., bFGF, can be used at a concentration of about 1 ng/mL to about 100 ng/mL, or more preferably, about 10 ng/mL to about 100 ng/mL. In one embodiment, the FGF is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL The BMP4 can be used at a concentration of about 1 ng/mL to about 100 ng/mL, or more preferably, about 10 ng/mL to about 100 ng/mL. In one embodiment, the BMP4 is used at a concentration of about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or 100 ng/mL. The small molecule inhibitor of transforming growth factor-beta (TGF-β) type I receptor, e.g., SB431542, can be used at a concentration of about 0.1 μM to about 100 μM, or more preferably, about 1 μM to about 100 μM. In one embodiment, the small molecule inhibitor of transforming growth factor-beta (TGF-β) type I receptor is used at a concentration of about 0.1 μM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM. The Forskolin can be used at a concentration of about 0.1 μM to about 10 μM. In one embodiment, the Forskolin is used at a concentration of about 0.1 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM.

In one embodiment, the VEGF is used at a concentration of about 50 ng/mL, the FGF is used at a concentration of about 50 ng/mL, the BMP4 is used at a concentration of about 25 ng/mL, the small molecule inhibitor is used at a concentration of about 10 and the Forskolin is used at a concentration of about 2 μM.

The second step of differentiation of mesoderm cells into meso-VPCs using the 2D differentiation platform may be performed under a hypoxia condition of about 5% CO₂ and about 5% O₂, or other known conditions suitable for the differentiation into vascular progenitor cells.

The two-step differentiation of mesoderm cells into meso-VPCs using the 2D differentiation platform may be conducted in any suitable containers known in the art. Exemplary tissue culture containers include, but are not limited to, 15 cm tissue culture plates, 10 cm tissue culture plates, 3 cm tissue culture plates, 6-well tissue culture plates, 12-well tissue culture plates, 24-well tissue culture plates, 48-well tissue culture plates, 96-well, tissue culture plates, T-25 tissue culture flasks, and T-75 tissue culture flasks. In one embodiment, the differentiation of mesoderm cells into meso-VPCs using the 2D differentiation platform is conducted in a T-75 tissue culture flask.

The differentiation of mesoderm cells into meso-VPCs using the 2D differentiation platform may be conducted on any suitable surface. In one embodiment, the differentiation of mesoderm cells into meso-VPCs using the 2D differentiation platform is conducted on an extracellular matrix surface. In one embodiment, the extracellular matrix surface is a collagen IV-coated surface.

In one embodiment, the meso-VPCs produced by the 2D differentiation platform may be further dissociated into single cells by enzymatic treatment.

In some embodiments of the invention, the mesoderm cells or meso-VPCs produced in each step may be further sorted by methods known in the art, e.g., flow cytometry, to select cells with certain expression profiles of molecule markers, e.g., cell-surface markers or miRNA markers. Methods of charactering the cells produced by the methods of the invention are further provided below.

### III. Characteristics and Compositions of Meso-VPCs

The present invention provides mesoderm-derived vascular progenitor cells (meso-VPCs) obtained by in vitro differentiation of mesoderm cells derived from pluripotent stem cells using the methods disclosed herein. In one embodiment, the pluripotent stem cells are first differentiated into mesoderm cells which, in turn, are differentiated into meso-VPCs. Expression levels of certain phenotypic markers may be determined by any method known in the art, such as flow cytometry/fluorescence-activated cell sorting (FACS), single cell mRNA profiling, or immunohistochemistry. Expression of certain genes may be determined by any method known in the art, such as RT-PCR and RNA-Seq.

In one embodiment, the population of meso-VPCs of the invention express at least 1, at least 2, at least 3, at least 4, at least 5, at least 6, at least 7, or at least 8 markers selected from the group consisting of CD31/PECAM1, CD309/KDR, CD43, CD144, CD34, CD184/CXCR4, CD146, and PDGFRb. In one embodiment, the population of meso-VPCs express CD31/PECAM1, CD309/KDR and CD146. In another embodiment, the population of meso-VPCs express CD31/PECAM1, CD309/KDR, CD146, and (i) at least one of CD144, CD34, CD184/CXCR4, CD43, or PDGFRb, (ii) CD34, CD184/CXCR4, and PDGFRb; (iii) CD184/CXCR4; (iv) PDGFRb; (v) CD144 and CD184/CXCR4; (vi) CD184/CXCR4 and CD43; or (vii) CC184/CXCFR4. In an embodiment, the population of meso-VPCs are considered expressing a certain marker if at least about 20% of the meso-VPCs in a composition express the marker.

In any of the embodiments, the population of meso-VPCs show limited or no detection of one or more of, CXCR7, CD45, and NG2. In any of the embodiments, the population of meso-VPCs exhibit limited or no detection of all of CXCR7, CD45, and NG2. In any of the embodiment, the population of meso-VPCs exhibit limited or no detection of one or more of CD144, CD34, CD184/CXCR4, CXCR7, CD43, CD45, PDGFRb, or NG2. In an embodiment, the population of meso-VPCs are considered exhibiting limited or no detection of a marker if less than about 20% of the meso-VPCs in a composition express the marker.

In one embodiment, at least about 20%, 25%, 30%, 35%, 40%, 45%, 50%, 55%, 60%, 65%, 70%, 75%, 80%, 85%, 90%, 95%, 98%, 99%, or 100% of the meso-VPCs in a composition express at least 1, at least 2, at least 3, at least 4, at least 5, at least 6, at least 7, or at least 8 markers selected from the group consisting of CD31/PECAM1, CD309/KDR, CD43, CD144, CD34, CD184/CXCR4, CD146, and PDGFRb. In one embodiment of the instant invention at least about 20%, 25%, 30%, 35%, 40%, 45%, 50%, 55%, 60%, 65%, 70%, 75%, 80%, 85%, 90%, 95%, 98%, 99%, or 100% of the meso-VPCs in a composition of the invention express CD31/PECAM1, CD309/KDR, and CD146. In one embodiment of the instant invention at least about 20%, 25%, 30%, 35%, 40%, 45%, 50%, 55%, 60%, 65%, 70%, 75%, 80%, 85%, 90%, 95%, 98%, 99%, or 100% of the meso-VPCs in a composition of the invention express CD31/PECAM1, CD309/KDR, CD146, and (i) at least one of CD144, CD34, CD184/CXCR4, CD43, or PDGFRb, (ii) CD34, CD184/CXCR4, and PDGFRb; (iii) CD184/CXCR4; (iv) PDGFRb; (v) CD144 and CD184/CXCR4; (vi) CD184/CXCR4 and CD43; or (vii) CC184/CXCFR4.

In any of the embodiments, less than about 20%, 15%, 10%, 5%, 4%, 3%, 2% or 1% of the meso-VPCs in a composition of the invention express one or more of CXCR7, CD45, and NG2. In any of the embodiments, less than about 20%, 15%, 10%, 5%, 4%, 3%, 2% or 1% of the meso-VPCs in a composition of the invention express all of CXCR7, CD45, and NG2. In any of the embodiments, less than about 20%, 15%, 10%, 5%, 4%, 3%, 2% or 1% of the meso-VPCs in a composition of the invention express one or more of CD144, CD34, CD184/CXCR4, CXCR7, CD43, CD45, PDGFRb, or NG2.

The meso-VPCs of the invention may be further characterized by single cell miRNA profiles. In one embodiment, the meso-VPCs of the invention are positive for at least 1, at least 2, at least 3, or at least 4 miRNA markers selected from the group consisting of mir126, mir125a-5p, mir24, and mir483-5p. In any of the embodiments, the meso-VPCs are negative for at least 1, at least 2, at least 3, at least 4, at least 5, at least 6, at least 7, at least 8, or at least 9 of the markers selected from the group consisting of mir367, mir302a, mir302b, mir302c, mirLet7-e, mir223, mir99a, mir142-3p, and mir133a. In one embodiment, the meso-VPCs are positive for mir126, mir125a-5p, mir24, and mir483-5p. In another embodiment, the meso-VPCs are positive for mir483-5p.

In one embodiment, about 50%, 55%, 60%, 65%, 70%, 75%, 80%, 85%, 90%, 95%, 98%, 99%, or 100% of the meso-VPCs in a composition are positive for at least 1, at least 2, at least 3, or at least 4 miRNA markers selected from the group consisting of mir126, mir125a-5p, mir24, and mir483-5p. In one embodiment of the instant invention at least 50%, 55%, 60%, 65%, 70%, 75%, 80%, 85%, 90%, 95%, 98%, 99%, or 100% of the meso-VPCs in a composition are positive for at least 1, at least 2, at least 3, or at least 4 markers selected from the group consisting of mir126, mir125a-5p, mir24, and mir483-5p. In any of the embodiments, less than about 50%, 45%, 40%, 35%, 30%, 25%, 20%, 15%, 10%, 5%, 4%, 3%, 2%, or 1% of the meso-VPCs in a composition express at least 1, at least 2, at least 3, at least 4, at least 5, at least 6, at least 7, at least 8, or at least 9 of the markers selected from the group consisting of mir367, mir302a, mir302b, mir302c, mirLet7-e, mir223, mir99a, mir142-3p, and mir133a. In one embodiment of the instant invention at least 50%, 55%, 60%, 65%, 70%, 75%, 80%, 85%, 90%, 95%, 98%, 99%, or 100% of the meso-VPCs in a composition are positive for mir126, mir125a-5p, mir24, and mir483-5p. In another embodiment, at least 50%, 55%, 60%, 65%, 70%, 75%, 80%, 85%, 90%, 95%, 98%, 99%, or 100% of the meso-VPCs in a composition are positive for mir483-5p.

In one embodiment, the population of meso-VPCs expresses at least 1, at least 2, at least 3, at least 4, at least 5, at least 6, at least 7, at least 8, at least 9, at least 10, at least 11, at least 12, at least 13, at least 14, or at least 15 miRNA markers selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p. In one embodiment, the population of meso-VPCs expresses hsa-miR-3917, hsa-miR-450a-2-3p, and hsa-miR-542-5p. In an embodiment, the population of meso-VPCs are considered expressing a certain marker if at least about 20% of the meso-VPCs in a composition express the marker.

In one embodiment, about 50%, 55%, 60%, 65%, 70%, 75%, 80%, 85%, 90%, 95%, 98%, 99%, or 100% of the meso-VPCs in a composition express at least 1, at least 2, at least 3, at least 4, at least 5, at least 6, at least 7, at least 8, at least 9, at least 10, at least 11, at least 12, at least 13, at least 14, or at least 15 miRNA markers selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p. In one embodiment of the instant invention at least 50%, 55%, 60%, 65%, 70%, 75%, 80%, 85%, 90%, 95%, 98%, 99%, or 100% of the meso-VPCs in a composition express at least 1, at least 2, at least 3, at least 4, at least 5, at least 6, at least 7, at least 8, at least 9, at least 10, at least 11, at least 12, at least 13, at least 14, or at least 15 miRNA markers selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p. In one embodiment, about 50%, 55%, 60%, 65%, 70%, 75%, 80%, 85%, 90%, 95%, 98%, 99%, or 100% of the meso-VPCs in a composition express hsa-miR-3917, hsa-miR-450a-2-3p, and hsa-miR-542-5p. In one embodiment, at least 50%, 55%, 60%, 65%, 70%, 75%, 80%, 85%, 90%, 95%, 98%, 99%, or 100% of the meso-VPCs in a composition express hsa-miR-3917, hsa-miR-450a-2-3p, and hsa-miR-542-5p.

In one embodiment, the population of meso-VPCs exhibits limited or no expression of at least 1, at least 2, at least 3, at least 4, at least 5, at least 6, or at least 7 miRNA markers selected from hsa-let-7e-3p, hsa-miR-99a-3p, hsa-miR-133a-5p, hsa-miR-11399, hsa-miR-196b-3p, hsa-miR-5690, and hsa-miR-7151-3p. In one embodiment, the population of meso-VPCs exhibits limited or no expression of hsa-let-7e-3p, hsa-miR-99a-3p, and hsa-miR-133a-5p. In an embodiment, the population of meso-VPCs exhibits limited or no expression of hsa-miR-11399, hsa-miR-196b-3p, hsa-miR-5690, and hsa-miR-7151-3p. In an embodiment, the population of meso-VPCs are considered exhibiting limited or no detection of a marker if less than about 20% of the meso-VPCs in a composition express the marker.

In one embodiment, about 50%, 55%, 60%, 65%, 70%, 75%, 80%, 85%, 90%, 95%, 98%, 99%, or 100% of the meso-VPCs in a composition exhibit limited or no expression of at least 1, at least 2, or at least 3 miRNA markers selected from hsa-let-7e-3p, hsa-miR-99a-3p, and hsa-miR-133a-5p. In one embodiment, at least 50%, 55%, 60%, 65%, 70%, 75%, 80%, 85%, 90%, 95%, 98%, 99%, or 100% of the meso-VPCs in a composition exhibit limited or no expression of at least 1, at least 2, or at least 3 miRNA markers selected from hsa-let-7e-3p, hsa-miR-99a-3p, and hsa-miR-133a-5p.

In addition to the characteristics described above, the meso-VPCs of the invention possess other properties of vascular progenitor cells, e.g., the potency of differentiating into vascular cells such as endothelial cells, smooth muscle cells, and hematopoietic cells. In one embodiment, the meso-VPCs of the invention possess the potency of differentiating into vascular endothelial cells. Other vascular cell properties of the meso-VPCs can be determined by, for example, Matrigel and AcLDL uptake assays.

In one embodiments, the meso-VPCs of the invention have morphology of vascular cells such as cobblestone endothelial-like morphology. Other methods of characterizing the meso-VPCs of the invention include karyotyping to determine the chromosomal integrity.

In an embodiment, the meso-VPCs of the invention are substantially purified with respect to pluripotent stem cells and mesoderm cells. In a further embodiment, meso-VPCs of the invention are substantially purified with respect to pluripotent stem cells and mesoderm cells such that said cells comprises at least about 70%, 75%, 80%, 85%, 90%, 91%, 92%, 93%, 94%, 95%, 96%, 97%, 98%, 99%, or 100% meso-VPCs. The pluripotent stem cells may be any pluripotent stem cells described herein.

The meso-VPCs may comprise less than about 30%, 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% pluripotent stem cells and mesoderm cells. The composition may be devoid of pluripotent stem cells and mesoderm cells.

### IV. Pharmaceutical Compositions Comprising Meso-VPCs

The present invention provides pharmaceutical compositions comprising any of the meso-VPCs described herein. Pharmaceutical compositions comprising meso-VPCs of the invention may be formulated with a pharmaceutically acceptable carrier. For example, meso-VPCs of the invention may be administered alone or as a component of a pharmaceutical formulation, wherein the meso-VPCs may be formulated for administration in any convenient way for use in medicine. Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions.

Other exemplary carriers or excipients are described, for example, in Hardman, et al. (2001) Goodman and Gilman's The Pharmacological Basis of Therapeutics, McGraw-Hill, New York, N. Y.; Gennaro (2000) Remington: The Science and Practice of Pharmacy, Lippincott, Williams, and Wilkins, New York, N. Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; and Weiner and Kotkoskie (2000) Excipient Toxicity and Safety, Marcel Dekker, Inc., New York, N. Y.

The pharmaceutical compositions comprising the meso-VPCs can be formulated in combination with one or more pharmaceutically acceptable sterile isotonic aqueous or non-aqueous solutions selected from the group consisting of dispersions, suspensions, emulsions, sterile powders optionally reconstituted into sterile injectable solutions or dispersions just prior to use, antioxidants, buffers, bactericides, solutes or suspending and thickening agents.

Exemplary pharmaceutical compositions of the present disclosure may be any formulation suitable for use in treating a human patient, such as a patient suffering from a vascular disease or disorder. In one embodiment, the pharmaceutical composition comprising meso-VPCs are formulated as an injectable material, e.g., a material suitable for intramuscular injection. The pharmaceutical composition comprising the meso-VPCs may be administered in a buffered solution at a physiological pH, further containing an osmotically active agent maintaining the solution at a physiologically osmolality. In one embodiment, the pharmaceutical composition comprising meso-VPCs may be administered in a buffer comprising at least 5% (w/v) glucose. In one embodiment, the pharmaceutical composition comprising meso-VPCs may be administered in a buffer comprising sodium chloride. Other reagents known in art can also be used to formulate the pharmaceutical composition. In one embodiment, the buffers or solutions used to formulate the pharmaceutical compositions are sterilized before use.

The pharmaceutical compositions comprising the meso-VPCs used in the methods described herein may be delivered in a suspension, gel, colloid, slurry, or mixture. Also, at the time of delivery, cryopreserved meso-VPCs may be resuspended with commercially available balanced salt solution to achieve the desired osmolality and concentration for administration by injection (e.g., bolus or intravenous). The pharmaceutical compositions comprising the meso-VPCs may be delivered, e.g., via one or more injections, to the subject in a mixture with a durable inert matrix. Durable inert matrix such as hydrogels—natural or synthetic water-insoluble polymers—could provide scaffolds for the cell's growth and expansion at the site of administration. In one embodiment, the pharmaceutical composition comprising the meso-VPCs is administered in a hyaluronan hydrogel. In one embodiment, the pharmaceutical composition comprising the meso-VPCs is administered in a methylcellulose hydrogel. Other suitable materials known in the art that provide durable inert matrix scaffolds for cell growth and expansion can also be used in the methods described herein.

The pharmaceutical compositions comprising the meso-VPCs may be delivered by one or more injections, e.g., via a syringe. Alternatively, the pharmaceutical compositions comprising the meso-VPCs may be delivered by other suitable methods known in the art. Suitable delivery methods may further facilitate the growth and survival of the meso-VPCs and prevent cell loss at the site of administration. In certain embodiments, appropriate delivery methods help retain the meso-VPCs at the site of the administration and provide optimal environment for cell growth. Accordingly, the pharmaceutical compositions comprising the meso-VPCs can also be formulated into, e.g., a hydrogel tube, a hydrogel sheet, a bioengineered patch made from natural or artificial materials, or a cell sheet that provides sufficient support for the meso-VPCs in the pharmaceutical composition. In one embodiment, the pharmaceutical compositions comprising the meso-VPCs are delivered in the form of a hydrogel tube. In one embodiment, the pharmaceutical compositions comprising the meso-VPCs are delivered in the form of a hydrogel sheet. In one embodiment, the pharmaceutical compositions comprising the meso-VPCs are delivered in the form of a bioengineered patch. In one embodiment, the pharmaceutical compositions comprising the meso-VPCs are delivered in the form of a cell sheet. Any other suitable methods known in the art can also be used in the delivery of the pharmaceutical compositions described herein.

Pharmaceutical compositions typically should be sterile and stable under the conditions of manufacture and storage. The compositions can be formulated as a solution, microemulsion, liposome, or other ordered structure. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the soluble factors may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

One aspect of the invention relates to a pharmaceutical composition suitable for use in a mammalian patient, e.g., a human patient, comprising at least 10⁴, 10⁵, 10⁶, 10⁷, 10⁸, 10⁹, 10¹⁰, 10¹¹, 10¹², or 10¹³ meso-VPCs and a pharmaceutically acceptable carrier.

Concentrations for administration of pharmaceutical preparations of meso-VPCs may be at any amount that is effective and, for example, substantially free of PSCs. For example, the pharmaceutical compositions may comprise the numbers and types of meso-VPCs described herein. In a particular embodiment, the pharmaceutical compositions of meso-VPC comprise about 1×10⁴ to about 1×10⁵, about 1×10⁵ to about 1×10⁶, about 1×10⁶ to about 1×10⁷, about 1×10⁷ to about 1×10⁸, about 1×10⁸ to about 1×10⁹, about 1×10⁹ to about 1×10¹⁰, about 1×10¹⁰ to about 1×10¹¹, about 1×10¹¹ to about 1×10¹², or about 1×10¹² to about 1×10¹³ of the meso-VPCs for systemic administration to a host in need thereof or about 1×10⁴ to about 1×10⁵, about 1×10⁵ to about 1×10⁶, 1×10⁶ to about 1×10⁷, about 1×10⁷ to about 1×10⁸, about 1×10⁸ to about 1×10⁹, about 1×10⁹ to about 1×10¹⁰, about 1×10¹⁰ to about 1×10¹¹, about 1×10¹¹ to about 1×10¹², or about 1×10¹² to about 1×10¹³ of said meso-VPCs for local administration to a host in need thereof.

### V. Methods of Treating Vascular Diseases

The meso-VPCs and pharmaceutical compositions comprising meso-VPCs described herein may be used for cell-based treatments. In particular, the instant invention provides methods for treating vascular diseases, e.g., critical limb ischemia. The methods include administering to a subject in need thereof, an effective amount of meso-VPCs, wherein the meso-VPCs are obtained by in vitro differentiation of mesoderm cells derived from pluripotent stem cells. In one embodiment, the pluripotent stem cells are differentiated into mesoderm cells which, in turn, are differentiated into meso-VPCs.

Vascular disease refers to any abnormal condition of the blood vessels (arteries and veins). Vascular diseases outside the heart can present themselves anywhere. The most common vascular diseases are stroke, peripheral artery disease (PAD), abdominal aortic aneurysm (AAA), carotid artery disease (CAD), arteriovenous malformation (AVM), critical limb ischemia (CLI), pulmonary embolism (blood clots), deep vein thrombosis (DVT), chronic venous insufficiency (CVI), and varicose veins. In one embodiment, the vascular disease is a peripheral artery disease (PAD). In one embodiment, the vascular disease is an ischemic disease, such as critical limb ischemia (CLI). In one embodiment, the vascular disease is atherosclerosis, peripheral artery disease (PAD), carotid artery disease, venous disease, blood clots, aortic aneurysm, fibromuscular dysplasia, lymphedema, or vascular injury. In one embodiment, the vascular disease is a periphery artery disease such as critical limb ischemia (CLI), intestinal ischemic syndrome, renal artery disease, popliteal entrapment syndrome, Raynaud's phenomenon, or Buerger's disease.

The meso-VPCs or pharmaceutical compositions may be used to treat any vascular diseases in a subject. In one embodiment, the meso-VPCs or pharmaceutical compositions are used to treat a periphery artery disease. In one embodiment, the meso-VPCs or pharmaceutical compositions are used to treat a periphery artery disease, including critical limb ischemia (CLI), intestinal ischemic syndrome, renal artery disease, popliteal entrapment syndrome, Raynaud's phenomenon, and Buerger's disease. In one embodiment, the meso-VPCs or pharmaceutical compositions are used to treat critical limb ischemia (CLI).

The meso-VPCs or pharmaceutical compositions of the instant invention may be administered systemically or locally. The meso-VPCs or pharmaceutical compositions may be administered using modalities known in the art including, but not limited to, injection via intravenous, intracranial, intramuscular, intraperitoneal, or other routes of administration, or local implantation, dependent on the particular pathology being treated. In one embodiment, the meso-VPCs or pharmaceutical compositions are administered intramuscularly.

The meso-VPCs or pharmaceutical compositions of the instant invention may be administered via local implantation, wherein a delivery device is utilized. Delivery devices of the instant invention are biocompatible and biodegradable. A delivery device of the instant invention can be manufactured using materials selected from the group comprising biocompatible fibers, biocompatible yarns, biocompatible foams, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biopolymers; homopolymers and copolymers of lactide, glycolide, epsilon-caprolactone, para-dioxanone, trimethylene carbonate; homopolymers and copolymers of lactide, glycolide, epsilon-caprolactone, para-dioxanone, trimethylene carbonate, fibrillar collagen, non-fibrillar collagen, collagens not treated with pepsin, collagens combined with other polymers, growth factors, extracellular matrix proteins, biologically relevant peptide fragments, hepatocyte growth factor, platelet-derived growth factors, platelet rich plasma, insulin growth factor, growth differentiation factor, vascular endothelial cell-derived growth factor, nicotinamide, glucagon like peptides, tenascin-C, laminin, anti-rejection agents, analgesics, anti-oxidants, anti-apoptotic agents anti-inflammatory agents and cytostatic agents.

The particular treatment regimen, route of administration, and adjuvant therapy may be tailored based on the particular pathology, the severity of the pathology, and the patient's overall health. Administration of the meso-VPCs or pharmaceutical compositions may be effective to reduce the severity of the manifestations of a pathology or and/or to prevent further degeneration of the manifestation of a pathology.

A treatment modality of the present invention may comprise the administration of a single dose of meso-VPCs or pharmaceutical compositions. Alternatively, treatment modalities described herein may comprise a course of therapy where meso-VPCs or pharmaceutical compositions are administered multiple times over some period of time. Exemplary courses of treatment may comprise weekly, biweekly, monthly, quarterly, biannually, or yearly treatments. Alternatively, treatment may proceed in phases whereby multiple doses are required initially (e.g., daily doses for the first week), and subsequently fewer and less frequent doses are needed.

In one embodiment, the meso-VPCs or pharmaceutical compositions are administered to a patient one or more times periodically throughout the life of a patient. In a further embodiment of the instant invention, the meso-VPCs or pharmaceutical compositions are administered once per year, once every 6-12 months, once every 3-6 months, once every 1-3 months, or once every 1-4 weeks. Alternatively, more frequent administration may be desirable for certain conditions or disorders. In one embodiment, the meso-VPCs or pharmaceutical compositions are administered via a device once, more than once, periodically throughout the lifetime of the patient, or as necessary for the particular patient and patient's pathology being treated. Similarly contemplated is a therapeutic regimen that changes over time. For example, more frequent treatment may be needed at the outset (e.g., daily or weekly treatment). Over time, as the patient's condition improves, less frequent treatment or even no further treatment may be needed.

In some embodiments, about 1×10⁴, about 1×10⁵, about 1.5×10⁵, about 2×10⁵, about 5×10⁵, about 1×10⁶, about 5×10⁶, about 10 million, about 20 million, about 40 million, about 60 million, about 80 million, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, about 500 million, about 520 million, about 540 million, about 560 million, about 580 million, about 600 million, about 620 million, about 640 million, about 660 million, about 680 million, about 700 million, about 720 million, about 740 million, about 760 million, about 780 million, about 800 million, about 820 million, about 840 million, about 860 million, about 880 million, about 900 million, about 920 million, about 940 million, about 960 million, or about 980 million meso-VPCs are administered into the subject. In some embodiments, about 1 billion, about 2 billion, about 3 billion, about 4 billion or about 5 billion meso-VPCs or more are administered. In some embodiments, the number of meso-VPCs ranges from between about 20 million to about 4 billion meso-VPCs, between about 40 million to about 1 billion meso-VPCs, between about 60 million to about 750 million meso-VPCs, between about 80 million to about 400 million meso-VPCs, between about 100 million to about 350 million meso-VPCs, and between about 175 million to about 250 million meso-VPCs.

The methods described herein may further comprise the step of monitoring the efficacy of treatment or prevention using methods known in the art. In one embodiment, the administration of the meso-VPCs or pharmaceutical compositions increases blood flow in the subject. In one embodiment, the administration of the meso-VPCs or pharmaceutical compositions promotes vascularization such as vasculogenesis and angiogenesis in the subject. In one embodiment, the administration of the meso-VPCs or pharmaceutical compositions reduces ischemic severity in the subject. In one embodiment, the administration of the meso-VPCs or pharmaceutical compositions reduces necrosis areas in the subject. Other physical and functional changes in the subject can also be measured and quantified to determine the efficacy of the methods of treatment of vascular diseases.

### VI. Kits

In some embodiments, the present invention provides kits comprising, in one or more separate compartments, the meso-VPCs or the pharmaceutical compositions of the invention. The kits may further comprise additional ingredients, e.g., gelling agents, emollients, surfactants, humectants, viscosity enhancers, emulsifiers in one or more compartments. The kits may optionally comprise instructions for formulating the meso-VPCs or the pharmaceutical compositions for diagnostic or therapeutic applications. The kits may also comprise instructions for using the components, either individually or together, in the therapy of vascular disorders and/or diseases. In one embodiment, the kit of the present invention includes a syringe for the injection of the pharmaceutical compositions comprising the meso-VPCs.

In some embodiments, the present invention provides kits comprising the meso-VPCs of the invention along with reagents for selecting, culturing, expanding, sustaining, and/or transplanting the meso-VPCs. Representative examples of cell selection kits, culture kits, expansion kits, transplantation kits are known in the art. Cells may also be enriched in the sample by using positive selection, negative selection, or a combination thereof for expression of gene products thereof.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1: Culturing of Human Pluripotent Stem Cells and Differentiation into Mesoderm Cells

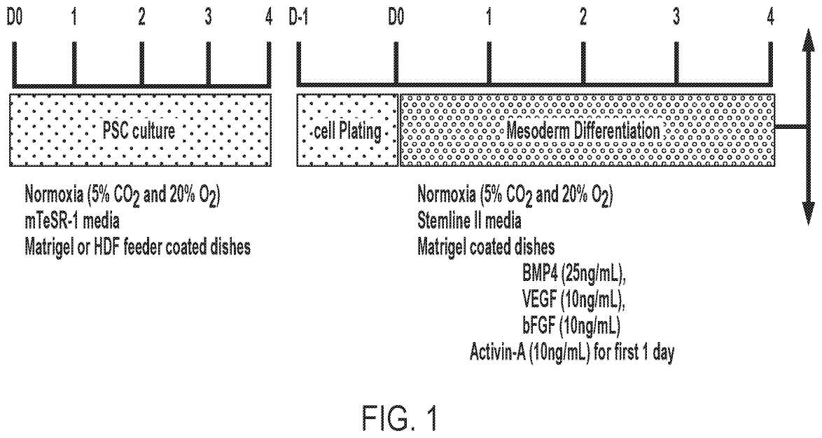

Proprietary human embryonic stem cell (hES) line, J1, and human induced pluripotent stem cell (hiPS) line, GMP1 were used in these studies. Cells were maintained in mTeSR1 complete media (Stem Cell Technologies) in 6 well tissue culture plates that were pre-coated with Matrigel (Corning) for feeder free culture conditions (hereafter called “FF”) or with Matrigel plus human dermal fibroblast or HDF for feeder culture conditions (hereafter called “HDF”) at 37° C. with 5% CO₂ plus 20% O₂ normoxia condition (FIG. 1). Media change was performed on days 1, 2, and 3 after plating of the cells (Day 0). Cells were passaged on Day 4 or when cell confluency reached 60-70%. For passaging, 1 mL/well of Dispase (1 U/ml, STEMCELL Technologies) was used for FF-cultured human pluripotent stem cells or 1 ml/well of cell dissociation buffer (CDB) (Gibco) was used for HDF-cultured human pluripotent stem cells. Cells were incubated for 5-7 minutes at 37° C. or until the edges of the colonies lifted from the plate. Dispase or CDB-containing media was carefully aspirated off from the plate and cells were gently washed with DMEM-F12 (Gibco) to remove any residual amount of enzyme or buffer. Fresh mTeSR1 complete media was then used to collect colonies from the plate using a forceful wash and scraping with a disposable cell scraper taking care to avoid formation of air bubbles, followed by centrifugation at 300×g for 5 minutes at room temperature (RT) to obtain a cell pellet. Following the removal of the supernatant, the cell pellet was re-suspended in the mTeSR1 complete media, and 1 mL of this homogenously mixed cell suspension was added in each well of 6 well tissue culture plates (pre-coated with Matrigel for FF culture or with Matrigel plus HDF as described above) containing 2 mL of mTeSR1 complete medium. Approximately 0.5 million cells in small cell clumps for FF culture or 0.25 million cells in small cell clumps for HDF culture were evenly distributed in each well. Cells were then spread out within the well using multiple side-to-side shaking motions while avoiding any swirling. Cultures were checked daily for growth quality and morphology.

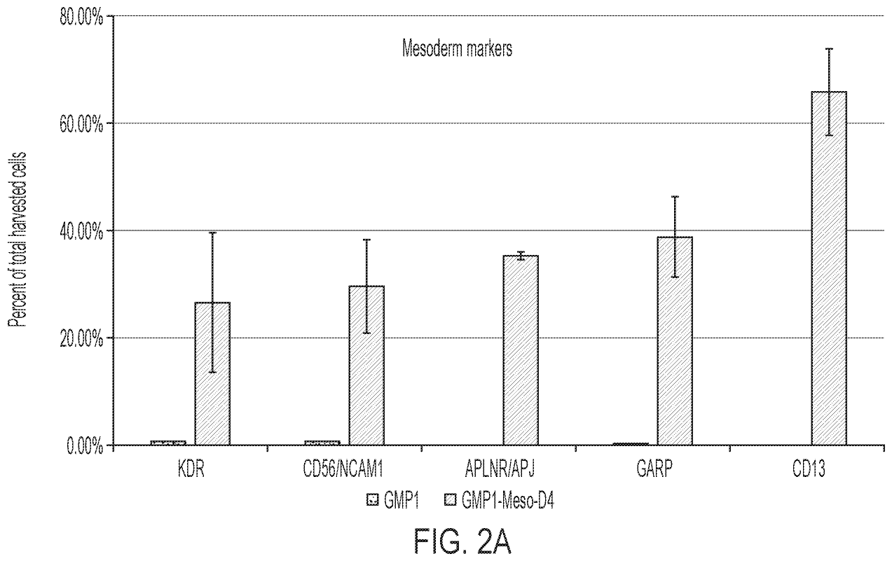

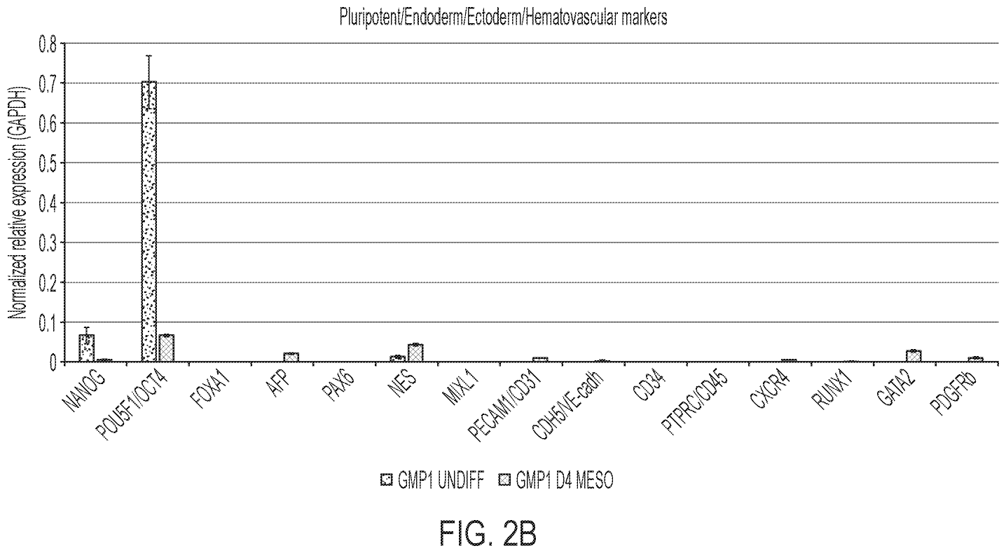

For differentiation of the pluripotent stem cells to mesoderm cells, Matrigel pre-coated 10-cm tissue culture dishes (Corning) were prepared by adding 5 mL Matrigel/dish. 10 mL mTeSR1 complete media/10-cm dish was immediately added after removal of unattached Matrigel from each dish to avoid drying out the Matrigel coated surfaces. Each Matrigel pre-coated 10-cm dish was seeded with approximately 1.5 million cells in small cell clumps from FF or HDF-cultured GMP1 cell culture (or approximately 300,000 cells/10-cm dish in small cell clumps from HDF-cultured J1 cell culture) evenly distributed in 10 mL TeSR1 complete media. Cells were then spread out within the dish using multiple side to side shaking motions while avoiding any swirling, and the plates were incubated for the next 24 hours (D-1) at 37° C. with 5% CO₂ plus 20% O₂ normoxia condition (FIG. 1). At D0 of differentiation, mTeSR1 complete media was replaced with 12 mL/10-cm dish Stemline II media (Sigma) containing a cocktail of mesoderm inducing growth factors, Activin A (10 ng/mL; Humanzyme), FGF-2 (10 ng/mL; Humanzyme), VEGF165 (10 ng/mL, Humanzyme), and BMP4 (25 ng/mL, Humanzyme). At D1 of differentiation, Activin-A was removed from the mesoderm cocktail, and media was replaced with 12 mL/dish fresh Stemline II media containing FGF-2 (10 ng/mL), VEGF165 (10 ng/mL), and BMP4 (25 ng/mL) to promote mesoderm cell emergence and expansion. Final media change was made at D3 of differentiation by adding 15 mL/dish fresh Stemline II media containing FGF-2 (10 ng/mL), VEGF165 (10 ng/mL), and BMP4 (25 ng/mL). Culture was continued until day 4, always at 37° C. with 5% CO₂ plus 20% O₂ normoxia condition (FIG. 1). Cells were then harvested by dissociating them into single cells using Stempro Acutase enzyme (Gibco). Cell characterization (by FACS and q-PCR analysis) was performed to confirm the presence of mesoderm characteristics of D4 harvested cells (FIGS. 2A-B).

### Example 2: Differentiation of Human Mesoderm Cells to Vascular Progenitor Cells (MESO-VPCs) Through a 3D-Vasclonoid Differentiation Platform

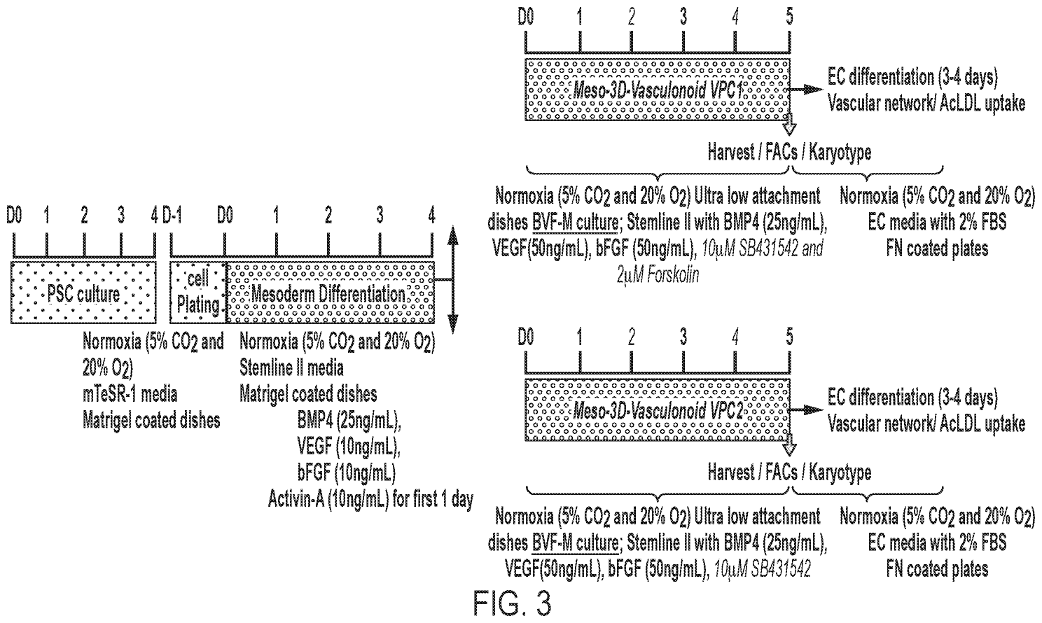

A novel 3D vasculonoid differentiation platform was developed by suspending the mesoderm cells obtained in Example 1 in VPC differentiation media using ultra-low attachment tissue culture dishes (Corning) in the presence of factors that promote vascular progenitor cell emergence and expansion (FIG. 3). At D0, 1 million unsorted D4 mesoderm cells were suspended in each well of ultra-low attachment 6 well plate and differentiated in VPC 3D differentiation media (Stemline II media containing 50 ng/mL VEGF165, 50 ng/mL FGF-2, 25 ng/mL BMP4 10 μM SB431542 and with 2 μM Forskolin (“Meso-3D Vasculonoid VPC1” protocol) or without Forskolin (“Meso-3D Vasculonoid VPC2” protocol) in a normoxia (37° C. with 5% CO₂ and 20% O₂). Respective media was changed at D2 and D4 and differentiation culture was completed at day 5. After 5 days of differentiation, MESO-VPCs from both protocols were harvested by dissociating them into single cells using Stempro Acutase enzyme. Cells were then counted and viability was measured followed by cryopreservation.

### Example 3: Differentiation of Human Mesoderm Cells to Vascular Progenitor Cells (MESO-VPCs) Through a 2D-Differentiation Platform

A novel 2D-based VPC differentiation platform was also developed by seeding the mesoderm cells produced according to Example 1 onto an adherent human extracellular matrix (collagen IV coated tissue culture dishes). At D0, 1.2 million unsorted D4 mesoderm cells (from above) were seeded onto human Collagen IV-coated (5 mg/cm2) T-175 flasks (Corning) and differentiated in VPC 2D differentiation media using two different (Meso-2D VPC2 and Meso-2D VPC3) differentiation protocols (FIG. 4). For the Meso-2D VPC2 protocol, Stemline II media containing 50 ng/mL VEGF165, 50 ng/mL FGF-2 and 25 ng/mL BMP4 was used at D0 (40 mL/flask), and Stemline II media containing 50 ng/mL VEGF165, 50 ng/mL FGF-2, 25 ng/mL BMP4 plus 10 μM SB431542 was used from D1 (45 mL/flask) through D7. For the Meso-2D VPC3 protocol, Stemline II media containing 50 ng/mL VEGF165, 50 ng/mL FGF-2, 25 ng/mL BMP4 and 2 μM Forskolin was used at D0, and Stemline II media containing 50 ng/mL VEGF165, 50 ng/mL FGF-2, 25 ng/mL BMP4 and 2 μM Forskolin plus 10 μM SB431542 was used from D1 through D7. D0 cells were cultured in a normoxia condition (37° C. with 5% CO₂ and 20% O₂). D1-D7 cells were cultured in a hypoxia condition (37° C. with 5% CO₂ and 5% O₂) with media changes performed at D3 (50 mL/flask) and D5 (60 mL/flask) of differentiation. After 7 days of differentiation, MESO-VPCs from the Meso-2D VPC2 and Meso-2D VPC3 protocols were harvested as single cells by enzymatic dissociation using the Stempro Acutase enzyme. Cells were then counted and viability was measured followed by cryopreservation as described in Example 2.

### Example 4: Matrigel/AcLDL Assay

Cryopreserved Meso-VPCs from Examples 2-3 were quickly thawed in a 37° C. water bath (2-3 minutes). The cells were then transferred into a 15 mL conical tube with 10 mL of endothelial cell or EC medium (VascuLife® VEGF medium from LifeLine Cell Technology) and centrifuged at 300×g for 5 minutes. After centrifugation, the supernatant was removed and cells were resuspended in 1 mL of fresh EC media for cell count. Cells were counted using Trypan blue and K2 Cellometer from Nexcelom Bioscience. A total of 18 mL cell suspension was prepared with a concentration of 10,000-20,000 viable MESO-VPCs/mL using EC medium. 3 mL/well of this cell suspension was plated onto fibronectin (FN) coated 6 well plates for 3-4 days in normoxia condition (37° C. with 5% CO₂ and 20% O₂) to prepare cells for Matrigel and AcLDL uptake assays).

For Matrigel/AcLDL assay, 250 μL basement membrane Matrigel (Corning) was added to each well of Nunc™ 4 well plates (Thermo Scientific) and the plates were incubated for 30 minutes at RT. Once the plates were coated the cells were seeded at a density of 5.0×10⁴ cells in 250 μL EC media per well. After 2-3 hours of plating, the media were replaced with fresh 250 μL EC media containing AcLDL (Molecular Probes) (5 μL AcLDL plus 245 μL EC media). Plates were incubated overnight in a normoxia condition. After 24 hours of incubation, AcLDL-containing media was removed, the plates were washed with D-PBS 3 times, and fresh 250 μL EC media/well was added. Finally, photomicrographs were taken from each well at ×4 magnification using Keyence Microscope.

### Example 5: Flow Cytometry Assay

Cryopreserved vials of day 5 harvested Meso-3D Vasculonoid VPC and day 7 harvested Meso-2D VPC (from Examples 2-3 above) were thawed and prepared into a single cell suspension in EC medium for cell count. Cells were resuspended in FACS buffer (D-PBS containing 2% FBS) after cell count was performed. Aliquots of the cells (100,000-200,000 cells/FACS assay sample) were prepared in 100 μL FACS buffer for surface marker antibody staining. Anti-human CD31/PECAM1 (BioLegend), CD34 (BD Biosciences), CD144/VE-Cadh (BioLegend), CD309/KDR (BioLegend), CD43 (BD Biosciences), CD45 (BD Biosciences), CD184/CXCR4 (BD Biosciences), CXCR7 (BioLegend), CD146 (BioLegend), NG2 (BD Biosciences), and PDGFRb (BioLegend) monoclonal antibodies were used at 5 μL/sample in 100 μL total volume. Cells were incubated with antibodies for 20-30 minutes on ice. After incubation, cells were washed to remove the unbound antibodies with 1 mL of FACS buffer. Cells were then centrifuged at 300×g for 5 minutes, the supernatant was removed, followed by resuspension in fresh 100 μL of FACS buffer with Propidium Iodide (PI, Sigma) at 1:1000 dilution. PI was added to the cell suspension and used for exclusion of dead cells during FACS analysis. The SONY SA3800 Spectral Analyzer was used for analysis. Compensation was set by using positive (HUVECs) and negative (undifferentiated J1 or GMP1 cells) controls.

### Example 6: Comparative Cells

For comparison with the Meso-VPCs of the invention, hemogenic endothelial cells (HE) and hemangioblasts (HB) were generated from human embryonic stem cells (e.g., J1 hESCs) or human induced pluripotent stem cells (e.g., GMP-1 iPSCs) using HE and HB protocols as previously described, for example, in U.S. Pat. Nos. 9,938,500; 9,410,123; WO 2013/082543; WO 2014/100779; U.S. Pat. No. 9,993,503; and U.S. Provisional Application No. 62/892,712 (filed Aug. 28, 2019) and its PCT application claiming priority thereto, all of which are incorporated herein by reference in their entirety. Briefly, for HE generation, hESCs or iPSCs were dissociated with Gibco® Cell Dissociation Buffer (CDB) to obtain single cell aggregates. The cells were resuspended at a final density of 400,000 cells/10 mL in mTeSR™1 medium (STEMCELL Technologies) containing Y-27632 (Stemgent) at a final concentration of 10 μM. 10 mL of the cell suspension was transferred into a collagen IV-coated 10 cm plate (Day −1). The plates were placed in the normoxic incubator overnight. The next day (Day 0), the mTeSR™1/Y-27632 media was gently removed from each 10 cm plate and replaced with 10 mL of BVF-M media [Stemline® II Hematopoietic Stem Cell Expansion Medium (Sigma); 25 ng/mL BMP4 (Humanzyme); 50 ng/mL VEGF165 (Humanzyme); 50 ng/mL FGF2 (Humanzyme)]. The plates were incubated in a hypoxia chamber (5% CO₂/5% O₂) for 2 days. On Day 2, the media was aspirated and fresh 10-12 mL of BVF-M was added to each 10 cm plate. On Day 4, the media was again aspirated and fresh 10-15 mL of BVF-M was added to each 10 cm plate. On Day 6, the cells were harvested for transplantation and/or for further testing. The media was aspirated from each plate and the plates were washed by adding 10 mL of D-PBS (Gibco) and aspirating the D-PBS. 5 mL of StemPro Accutase (Gibco) was added to each 10 cm plate and incubated for 3-5 minutes in a normoxic CO₂ incubator (5% CO₂/20% O₂). The cells were pipetted 5 times with a 5 mL pipet, followed by a P1000 pipet about 5 times. The cells were then strained through a 30 μM cell strainer and transferred into a collection tube. Each of the 10 cm plates were again rinsed with 10 mL of EGM-2 medium (Lonza) or Stemline® II Hematopoietic Stem Cell Expansion Medium (Sigma) and the cells were passed through a 30 μM cell strainer and collected in the collection tube. The tubes were centrifuged at 120-250×g for 5 minutes. The cells were then resuspended with EGM-2 media or Stemline® II Hematopoietic Stem Cell Expansion Medium (Sigma) and counted. After counting, the cells were spun down (250×g for 5 minutes) and resuspended with Freezing medium (10% DMSO+Heat Inactivated FBS) at a concentration of 3×10⁶ cells/mL. To create frozen stocks, cell suspension was aliquoted in 2 mL FBS (Hyclone) and DMSO (Sigma) per cryovial (6×10⁶ cells/2 mL/vial).

For hemangioblast (HB) generation, hESCs or iPSCs were dissociated with 4 mg/mL collagenase IV (Gibco) to obtain cellular clumps and then resuspended in BV-M media [Stemline® II Hematopoietic Stem Cell Expansion Medium (Sigma); 25 ng/mL BMP4 (Humanzyme); 50 ng/mL VEGF165 (Humanzyme)] and plated onto Ultra Low Attachment Surface 6 well plates (Corning) at a density of about 750,000-1,200,000 cells per well. The plates were placed in an incubator for 48 hours in a normoxic CO₂ incubator to allow embryoid body formation (Days 0-2). The media and cells in each well were then collected and centrifuged at 120-300 g for 3 minutes. Half of the supernatant was removed and replaced with 2 mL BV-M containing 50 ng/mL bFGF. Therefore, the final concentration of bFGF in the cell suspension was about 25 mg/mL 4 mL of the cell suspension was plated onto each well of a Ultra Low Attachment Surface 6 well plates and placed into a normoxic CO₂ incubator for another 48 hrs (Days 2-4) to allow continued embryoid body formation. On Day 4, the embryoid bodies were collected into a 15 mL tube, centrifuged at 120-300×g for 2 minutes, washed with D-PBS, and disaggregated into single cell suspensions using StemPro Accutase (Gibco). FBS (Hyclone) was used to inactivate the Accutase and the single cells were passed through a cell strainer, centrifuged, and resuspended in Stemline II media (Sigma) at about 1×10⁶ cells/mL. About 3×10⁶ cells were mixed in 30 mL Methocult BGM medium [MethoCult™ SF H4536 (no EPO) (StemCell Technologies); penicillin/streptomycin (Gibco); ExCyte Cell Growth Supplement (1:100) (Millipore); 50 ng/mL Flt3 ligand (PeproTech); 50 ngm/ml VEGF (Humanzyme); 50 ng/mL TPO (PeproTech); 30 ng/mL bFGF (Humanzyme)], replated on Ultra Low Attachment Surface 10 cm dishes (Corning), and incubated in a normoxic CO₂ incubator for 7 days (Days 4-11) to allow for formation of hemangioblasts. On Day 11, the hemangioblasts were harvested for transplantation and/or for further testing. Hemangioblasts were collected by diluting the methylcellulose with D-PBS (Gibco). The cell mixture was centrifuged at 300×g for 15 minutes twice, and resuspended in 30 mL of EGM2 BulletKit media (Lonza) or StemlineII and the cells were counted and frozen as described above.

### Example 7A: The 3D Vasculonoid Differentiation Platform Generates Cells with Vascular Progenitor Properties

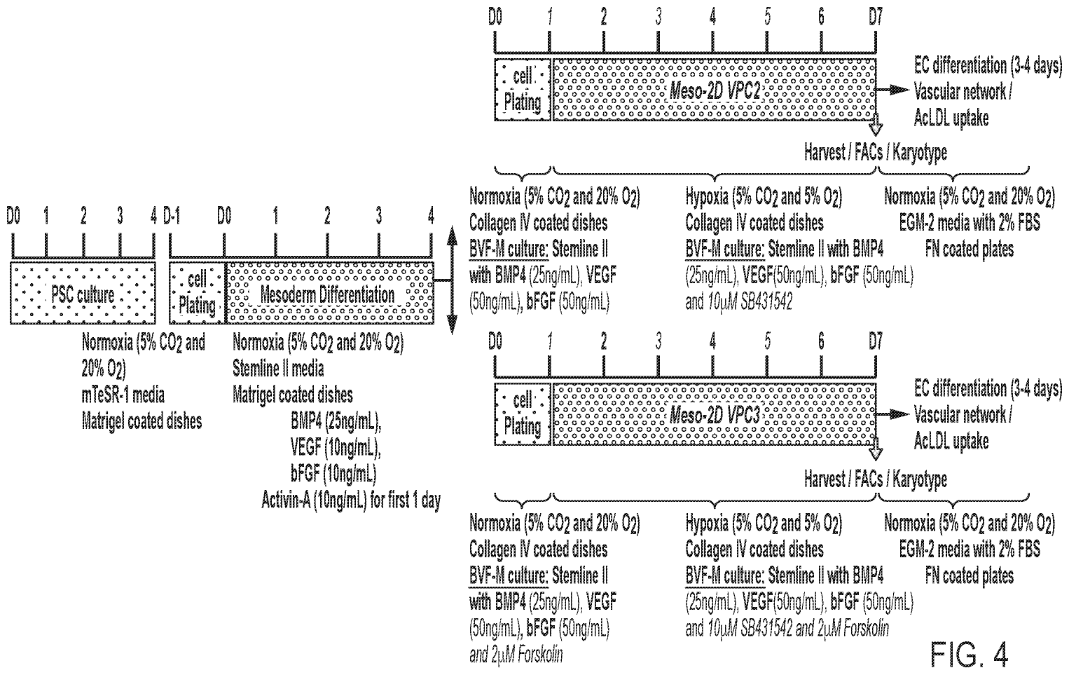

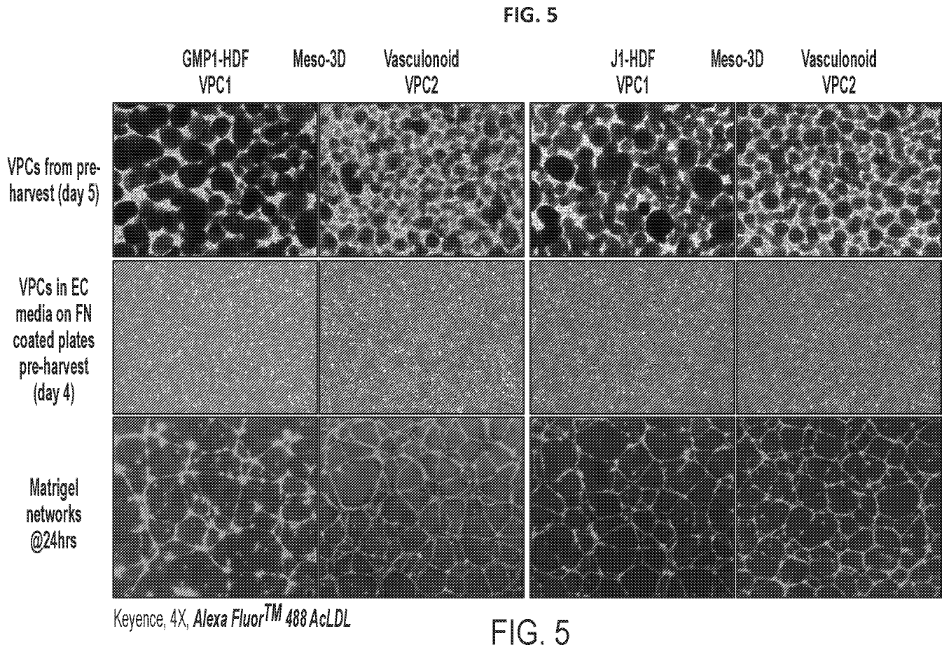

As described in Example 2, Meso-VPCs were generated using two different 3D differentiation protocols (Meso-3D Vasculonoid VPC1 and Meso-3D Vasculonoid VPC2) under normoxia condition (37° C. with 5% CO₂ and 20% O₂) for 5 days (FIG. 3). Seeded mesoderm cells remained viable and formed cell aggregates as early as day 1 (data not shown). These cell aggregates (hereafter called “Vasculonoid”) grew in size by day 5 (FIG. 5; top panels), at which point they were harvested. The Meso-3D Vasculonoid VPC1 protocol gave rise to bigger vasculonoid aggregates compared to the Meso-3D Vasculonoid VPC2 protocol. After cell harvest, the cells were re-plated in FN coated plate to determine their capacity to undergo further differentiation into endothelial lineage. As shown in FIG. 5; middle panels, when cells were cultured on FN coated plates in media that promote endothelial differentiation they acquired cobblestone morphology typical of endothelial cells. Meso-3D VPCs also exhibited robust capacity for the formation of capillary like networks on Matrigel, and displayed AcLDL uptake (FIG. 5; lower panels). VPC2 cells showed higher capacity for tube formation compared to VPC1 cells (FIG. 5; lower panels).

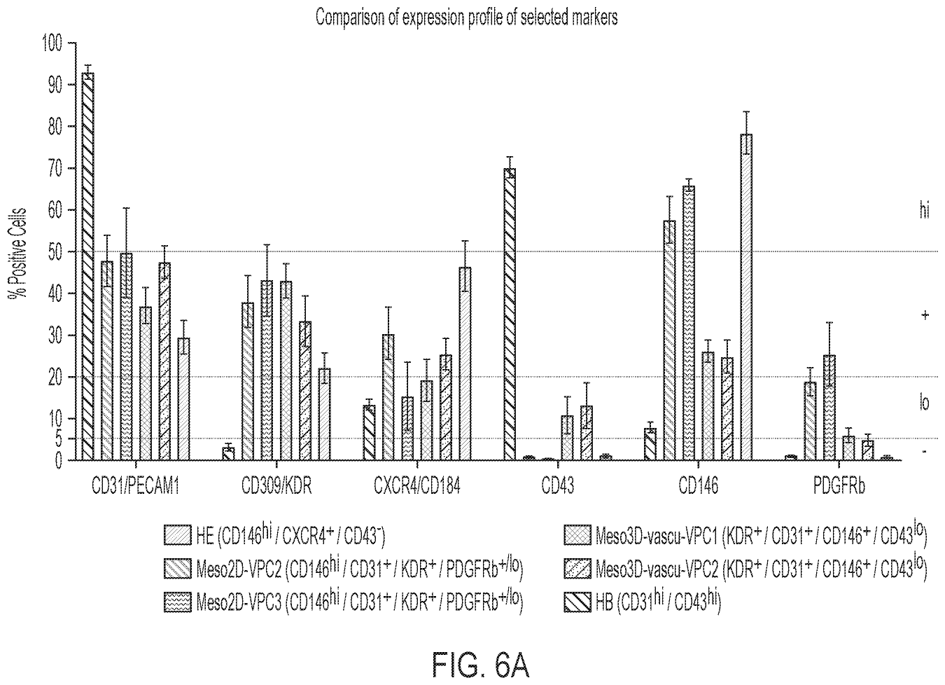

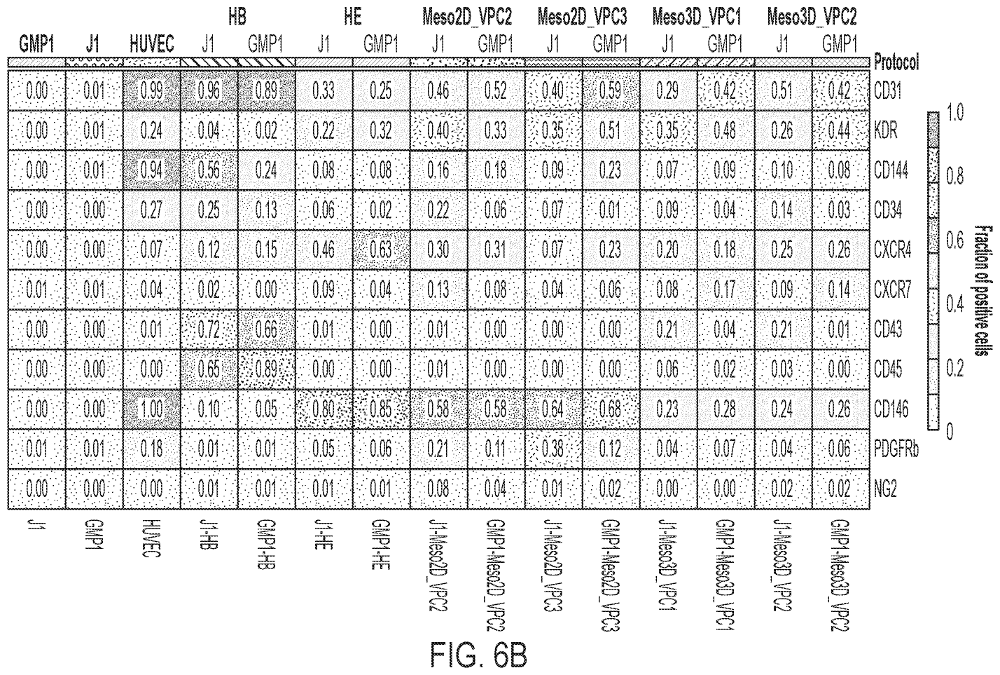

In addition, FACS analysis for vascular markers indicated that both J1 and GMP1-derived Meso-3D Vasculonoid VPC1 and VPC2 cells displayed robust expression (>20%) expression of endothelial markers KDR, CD31 as well as endothelial/pericyte (CD146) (FIG. 6A) and low expression of hematopoietic marker CD43. Their broad vascular marker expression profile was distinct from the expression profiles observed in undifferentiated pluripotent stem cells (GMP1 and J1) or HUVEC cells and those observed in other PSC derived cells (e.g. HB & HE) (FIGS. 6 B-C). Chromosomal stability of these differentiated cells was performed by G-banding karyotype analysis and the cells displayed normal karyotype indicating that differentiation of hES and hiPS cells through Meso-3D Vasculonoid VPC1 and Meso-3D Vasculonoid VPC2 protocols does not alter chromosomal stability during differentiation (data not shown).

### Example 7B: The 2D Differentiation Platform Generates Cells with Vascular Progenitor Properties

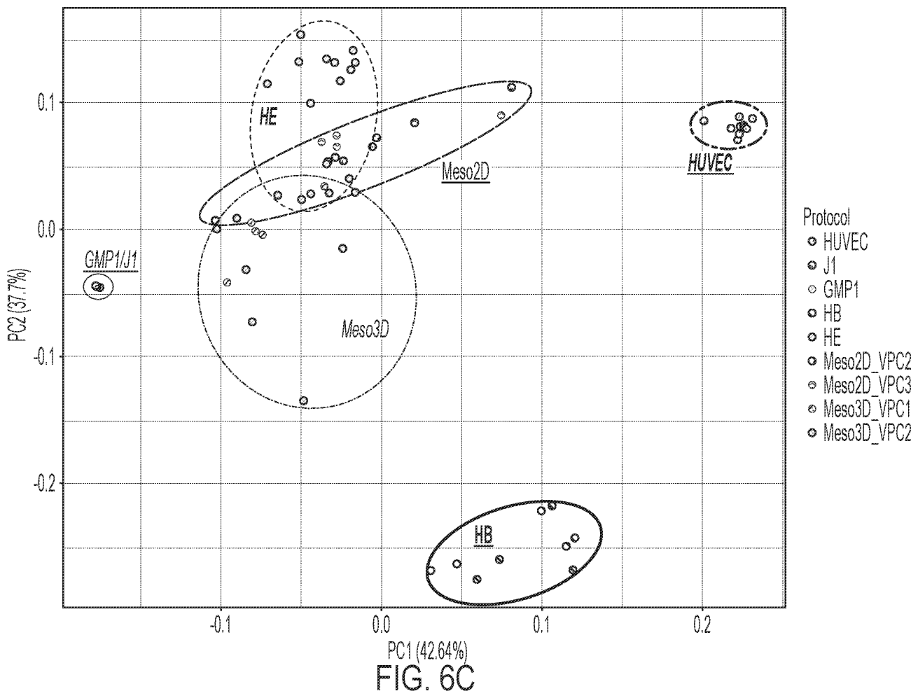

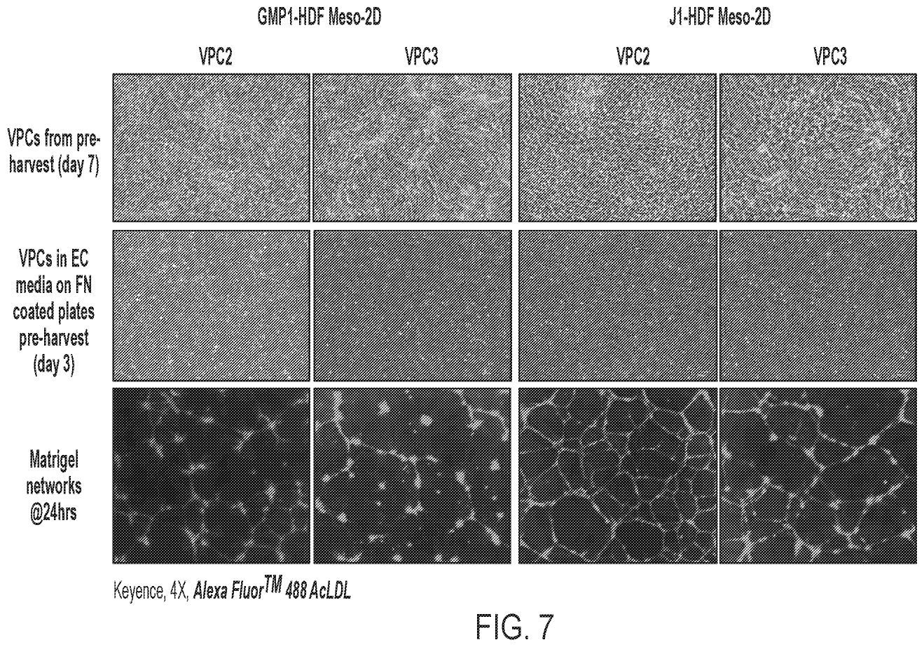

As described in Example 3, Meso-VPCs were generated using two different 2D differentiation protocols (Meso-2D VPC2 and Meso-2D VPC3) from iPS cells (GMP1) and hES cells (J1) under normoxia and hypoxia culture conditions for a total of 7 days (FIG. 4). The seeded mesoderm cells attached to the collagen IV coated surface, grew and expanded into bigger compact cell colonies by day 7 (harvest day) as a 2D differentiated adherent cell culture (FIG. 7 top panels). The Meso-2D VPC2 protocol gave rise to more compact cell colonies compared to the Meso-2D VPC3 protocol (colonies were more “spiky” or “swirly”) for both J1 and GMP1-derived cells. After cell harvest at day 7 and upon further culture on FN-coated plates and exposure to endothelial culture media, the cells exhibited typical vascular progenitor properties, including cobblestone endothelial-like morphology (FIG. 7; middle panels) and the capacity for formation of capillary like networks on Matrigel and AcLDL uptake (FIG. 7; bottom panels), however at lower scale than Meso-3D cells (comparison of FIG. 5 and FIG. 7 bottom panels).

In addition, FACS analysis for vascular markers indicated that both J1 and GMP1-derived Meso-2D VPC1 and VPC2 cells displayed high expression of CD146, robust expression (>20%) of endothelial markers KDR, CD31 and detectable (10-40%) expression of PDGFRb. This expression profile was distinct from the expression profiles observed in undifferentiated pluripotent stem cells or HUVEC cells and those observed in other PSC derived vascular progenitor cells (e.g. HB & HE) (FIGS. 6B and 6C). Compared to Meso-3D cells, Meso-2D VPCs expressed higher levels of CD146 and unique expression of PDGFR suggesting a higher propensity towards pericyte differentiation (FIGS. 6A-B). Moreover, unlike Meso-3Ds, Meso-2D VPCs did not express any of the blood markers CD45 or CD43.

### Example 8: Single Cell miRNA Profile

Additional analysis using single cell qRT-PCR analysis to evaluate the levels of expression of 96 microRNA associated with pluripotency or vascular cell identity was performed as described below. TaqMan Gene Expression Assays (Applied Biosystems) were ordered for 96 human miRNAs. 10× Assays were prepared by mixing 25 μL of 20× Taqman assays with 25 μL of 2× Assay Loading Reagent (Fluidigm) for a 50 μL volume of final stock. An aliquot of cells (frozen or freshly harvested) in the range of 66,000 to 250,000 cells/mL was prepared. The cells were incubated with LIVE/DEAD staining solution (LIVE/DEAD Viability/Cytotoxicity Kit) for 10 minutes at room temperature. The cells were then washed, suspended in media and filtered through a 40 μm filter. Cell counting was performed for viability and cell concentration using cellometer. A cell mix was prepared by mixing cells (60 μL) with suspension reagent (40 μL) (Fluidigm) in a ratio of 3:2. 6 μL of the cell suspension mix was loaded onto a primed C1 Single-Cell Autoprep IFC microfluidic chip for medium cells (10-17 μm) or large cells (17-25 μm), and the chip was then processed on the Fluidigm C1 instrument using the “STA: Cell Load(1782×/1783×/1784×)” script. This step captured one cell in each of the 96 capture chambers. The chip was then transferred to a Keyence Microscope and each chamber was scanned to score number of single cell captures, live/dead status of cells and doublet/cell aggregates captured. For Cell Lysis, Reverse Transcription, and Preamplification on the C1, Harvest reagent, Lysis final mix, RT final mix and Preamp mix were added to designated wells of the C1 chip according to manufacturer's protocol. The IFC was then placed in the C1 and “STA:miRNA Preamp (1782×/1783×/1784×) script was used. The cDNA harvest was programmed to finish the next morning. The cDNA was transferred from each chamber of the C1 chip to a fresh 96 well plate that was pre-loaded with 12.5 μL of C1DNA dilution reagent. Tube controls such as the no template control and the positive control were prepared for each experiment according to manufacturer's instructions. Preamplified cDNA samples were analyzed by qPCR using the 96.96 Dynamic Array™ IFCs and the BioMark™ HD System. Processing of the IFC priming in JUNO instrument followed by loading of cDNA sample mixes and 10× Assays was performed per manufacturer's protocol. The IFC was then placed into the Biomark™ HD system and PCR was performed using the protocol “GE96×96 miRNA Standard v1.pc1). Data analysis was performed using the Real-Time PCR Analysis software provided by Fluidigm. Dead cells, duplicates etc. were removed from analysis and the Linear Derivative Baseline and User Detector Ct Threshold based methods were used for analysis. The data were viewed in Heatmap view and exported as a CSV File. “R” software was then used to perform “Outlier Identification” analysis that resulted in a “FSO” file, and then instructions for “Automatic Analysis” were followed.

Results

Table 1: miRNA profile

As shown in Table 1, MESO-VPCs (either 3D or 2D) were negative for the pluripotent stem cells miRNA markers (mir376, mir302a, mir302b and mir 302c) and positive for endothelial miRNA markers expressed in HUVEC such as mir126, mir125a-5p and mir24. Still, MESO-VPCs were negative for the HUVEC specific miRNAs (mirLet7-e, mir223 and mir99a). Finally, MESO-VPCs showed unique expression of miRNA 483-5P and were negative for the HB and HE unique miRNAs mir142-3p and 133a, respectively.

### Example 9: In Vivo Study in a Hind-Limb Ischemia Model

Peripheral artery disease (PAD) is a form of peripheral vascular diseases (PVD) in which there are partial or total blockage of blood supply to a limb, usually the leg, leading to impaired blood flow and hypoxia in the tissue. When PAD advances, it reaches the stage of critical limb ischemia (CLI) with skin ulcerations, gangrene, and unavoidable amputations. Hind limb ischemia animal models have been used to evaluate various therapeutic approaches. In this study, a stable severe ischemia model (Ishikane et al. (2008) Stem Cells, 16:2625-2633) was used to assess the efficacy of meso-VPCs and to demonstrate improvement in blood flow restoration and signs of donor cell incorporation in the ischemic limb. The induction of hind limb ischemia in mice involves two ligations of the proximal end of the iliac and femoral arteries and its dissection between the two ligatures. The surgery causes obstruction of the blood flow and subsequently severe ischemic damage.

### Species

Mice/Balb/cOlaHsd-Foxn1^{nu} (Charles River Laboratories) aged 6-8 weeks at study initiation with minimum and maximum body weights within a range of +/−20% of the group mean weight.

### Test Articles

Test Item 1=J1-HDF Meso-2D VPC2 prepared according to Example 3

Test Item 2=J1-HDF Meso-3D Vasculonoid VPC2 prepared according to Example 2

Test Item 3=GMP-1-HDF Meso-2D VPC2 prepared according to Example 3

Test Item 4=GMP1-HDF Meso-3D Vasculonoid VPC2 prepared according to Example 2

Test Item 5=GMP1-HDF Meso-3D Vasculonoid VPC1 prepared according to Example 2

### Vehicle (Negative Control)

GS2 (cell-free medium as described in WO 2017/031312, which is incorporated herein by reference in its entirety) [for 552.2 mL of GS2: 0.9% Sodium Choride Irrigation USP (Baxter Healthcare or Hospira) (408.6 mL); 5% Dextrose/0.9% Sodium Chloride, Injection USP (Baxter or Braun) (33.2 mL), and BSS Irrigation Solution (Alcon) (110.4 mL)]

### Study Design and Timeline

The study was conducted according to the following study design (Table 2) and timeline (Table 3)

TABLE 2
Study Design

| | | Dose | Route of | | |
|---|---|---|---|---|---|
| | | Vol- | Adminis- | Number of animals | Termin- |
| Group | Treatment | ume | tration | (animal numbers) | ation |
| 1M | Sham- | N/A | N/A | (19, 20, 39, 40, 59, 60, | Day 36 |
| | operated | | | 79, 80, 99, 100, 101, | |
| | | | | 102, 103, 104, 105) | |
| 2M | Vehicle | 100 | IM | (13, 14, 15, 16, 17, 18, | |
| | | μl | | 54, 55, 56, 57, 58, | |
| | | | | 77, 78, 97, 98) | |
| 3M | Test | | IM | (61, 62, 63, 64, 65, 66, | |
| | Item 1 | | | 67, 68, 76, 90, 91, | |
| | | | | 92, 93, 94, 95, 96) | |
| 4M | Test | | IM | (69, 70, 71, 72, 73, | |
| | Item 2 | | | 74, 75, 81, 82, 83, 84, | |
| | | | | 85, 87, 88, 89) | |
| 5M | Test | | IM | (1,2, 3, 4, 5, 9, 10, 11, | |
| | Item 3 | | | 12, 21, 22, 23, 24, | |
| | | | | 25, 26) | |
| 6M | Test | | IM | (6, 7, 8, 36, 37, 38, | |
| | Item 4 | | | 45, 46, 47, 48, 49, 50, | |
| | | | | 51, 52, 53) | |
| 7M | Test | | IM | (27, 28, 29, 30, 31, | |
| | Item 5 | | | 32, 33, 34, 35, 41, 42, | |
| | | | | 43, 44) | |

IM = local intramuscular injection to the ischemic limb

TABLE 3
Timeline

| Study Day | Procedure | Sacrifice |
|---|---|---|
| Before treatment and once a | Body weight | |
| week thereafter | | |
| Day 0 | HLI surgery | |
| Day 0 | Test Item IM injection | |
| Before and after HLI surgery | Blood Flow measurement | |
| and on Days: 7, 14, 21, 2 | | |
| 8 and 35 | | |
| On Days: 7, 21and 35 | Blood vessel imaging | |
| On Days: 7, 14, 21, 28 and | Limb function and limb | |
| 35 | necrosis evaluation | |
| Twice a week | Clinical score | |
| On Day 35 | Clinical assessment | |
| | of limb necrosis (by | |
| | scale) | |
| On Day 36 | | Gastrocnemius |
| | | muscle |
| | | harvesting |

### Experimental Procedures

### Morbidity and Mortality Observation

Animals were monitored continuously during the surgery day and twice a day thereafter (once a day over the weekend).

### Body Weight

Body weight was recorded before treatment and once a week thereafter.

### HLI Surgery Procedure

Under anesthesia and analgesia, the mouse was placed with ventral side up.

On the day of surgery (Day 0) an incision was made in the skin in the inguinal area of the right hind-limb. The femoral artery was ligated twice with 6-0 silk thread and transected between the two ligatures. The wound was closed with 5-0 Vicryl absorbable thread and the mouse was allowed to recover.

### Test Item Administration Procedure

On Day 0 immediately post-surgery, each animal was injected intramuscular at two sites: the proximal and the distal sides of the surgical wound. The animals were injected 50 μl in each site, total 100 μl per animal. Total amount per mouse was 1 M cells/mouse.

### Blood Flow Measurement Procedure

Blood flow in both legs for each mouse was measured with a non-contact Peri-Med LASER Doppler before surgery, immediately after surgery and just before the treatment for inclusion criteria (only animals in which blood flow was reduced at least 30% compared to the uninjured leg was included) and on study Days: 7, 14, 21, 28, and 35 post operation. Blood flow measurements was expressed as the ratio of the flow in the ischemic limb to that in the normal limb after the surgery and as the ratio of the flow in the right limb to that in the left limb.

### Blood Vessel Imaging Procedure

Blood vessel imaging in both legs (in femoral and tibial areas) for 3 mice per group for 3 time-points (7, 21 and 35 days after surgery) was measured by RSOM Explorer P50″ (i-Thera Medical) imaging system. The RSOM (Raster Scanning Optoacoustic Mesoscopy) Explorer P50 works by illumination with nanosecond laser pulses at 532 nm and a spherically focused 50 MHz detector. An eighty second acquisition time allowed imaging of a field of view of 5×5 mm, penetration of 3 mm and at axial/lateral resolution of 40 μm/10 μm.

### Macroscopic Evaluation of Ischemic Severity Procedure

Macroscopic evaluation of the ischemic limb was done every week post operation started from Day 7 by using morphological grades for necrotic area according to Table 4 (see Goto et al. Tokai J. Exp. Clin. Med. 2006. 31:128-132).

TABLE 4
Morphological grades for necrotic area

| Grade | Description |
|---|---|
| 0 | absence of necrosis |
| 1 | necrosis limiting to toes (toes loss), |
| 2 | necrosis extending to a dorsum pedis (foot loss), |
| 3 | necrosis extending to a crus (knee loss) |
| 4 | necrosis extending to a thigh (total hind-limb loss) |

### In Vivo Assessment of Limb Function Procedure

Semi-quantitative assessment of impaired use of the ischemic limb was performed every week post operation started from Day 7 using the following scale in Table 5 (see Stabile et al. Circulation. 2003. 108:205-210).

TABLE 5
Assessments of limb function

| Grade | Description |
|---|---|
| 0 | flexing the toes to resist gentle traction of the tail |
| 1 | plantar flexion |
| 2 | no dragging but no plantar flexion |
| 3 | dragging of foot |

Limb function was graded as “Not applicable” or “N/A” in case of partial or full limb amputation. In such cases, blood flow measurements was not included in the statistical analysis.

### Animal Sacrifice and Tissue Fixation

On Day 36, mice were sacrificed. Gastrocnemius muscle from both hind-limbs were collected, fixed in formalin and embedded in paraffin (5 animals per group). From 3 animals per group muscle was OCT embedded, frozen and stocked for further shipment. Embedded muscle samples were sectioned, stained by H&E+IHC Isolectin B4-HRP conjugated and evaluated by a pathologist. IHC for human specific antibody (Stem 121) was performed for presence of human cells in tissues. ICH staining for CD34 and vascular density evaluation was performed.

### Results

### Mortality

Fourteen animals died during the study. Among them: one mouse died during the procedure. Thirteen mice were found dead in their cages within 11 days following HLI surgery. Among them mice numbers:19, 40, 99, 100 and 101 from the group 1M; mouse number 97 from the group 2M; mice numbers: 69, 72, 88 and 89 from the group 4M; mice numbers: 38 and 50 from the group 6M; mouse number 28 from the group 7. Twenty mice were euthanized by the humanistic reason due to legs amputation (mice numbers:58 and 98 from the group 2M; mice numbers: 61, 63, 64, 90, 91, 92, 93 and 95 from the group 3M; mouse number 81 from the group 4M; mouse number 21 from the group 5M; mice numbers:36, 37, 47 and 53 from the group 6M; mice numbers:29, 30, 32, and 34 from the group 7M. All animals that were surviving at each time point were evaluated at that time point.

### Body Weight

Body weight was monitored up to Study Day 35. The weight dropped during the first week after surgery, but started to recover during the second week to reach almost full recovery during the last week. All animal groups recovered in parallel. Two-way ANOVA followed by Bonferroni post-hoc comparisons performed using GraphPad Prism 5 software did not reveal statistically significant differences in body weight between all groups.

### Blood Flow Measurement

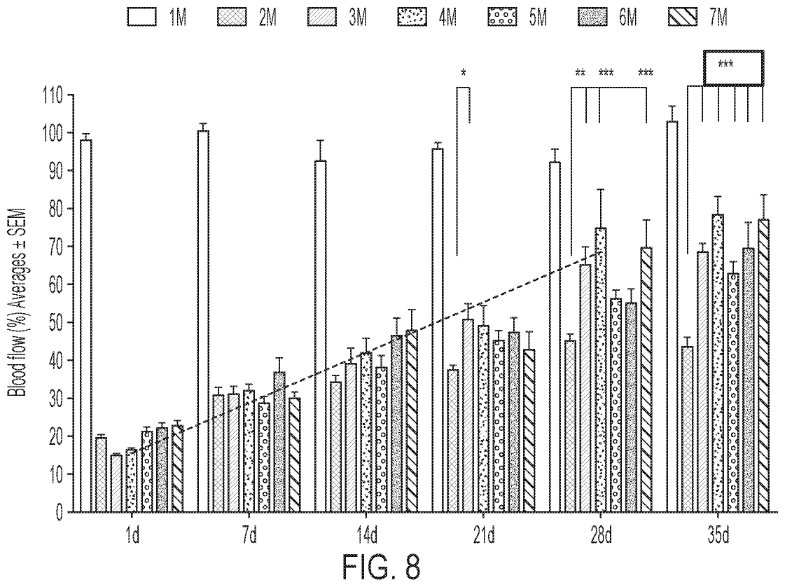

Blood flow was assessed prior to test item treatment and marked changes were observed in all the operated animals thereafter. Significant improvement in blood flow was observed throughout the study in all treated groups (3-7M) compared to the vehicle treated group (2M). This improvement was statistically significant (two-way ANOVA followed by Bonferroni multiple comparisons) for the right operated limb from Day 21 for group 3M and from Day 28 until Day 35 for the other treated groups (FIG. 8).

### Blood Vessel Imaging

Blood vessel imaging in both legs (at the femoral and tibial areas) for 3 mice per group for 3 time-points (7, 21 and 35 days after surgery) was measured using the RSOM Explorer P50 (i-Thera Medical) imaging system.

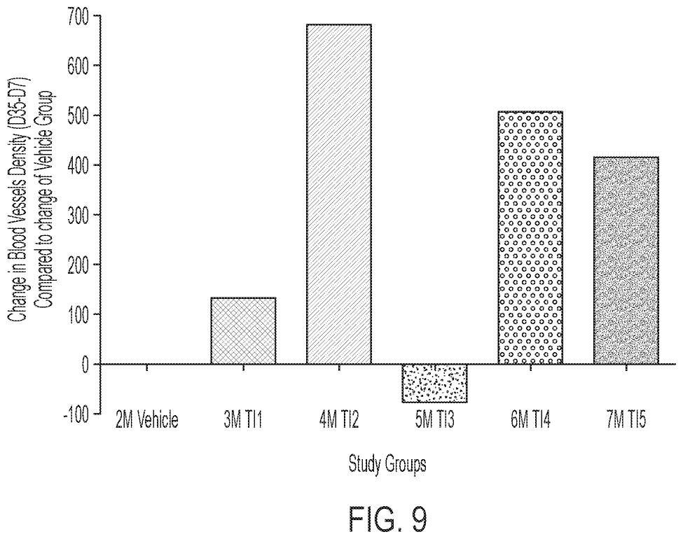

Several analysis methods were used to evaluate the possible increase in small blood vessels density in ischemic hind limb. Finally, integration of the 100 highest section was used as more reliable to see the blood vessels angiogenesis. Results were presented as summary of Day 35 as percent of Day 7. In order to make the data clearer, the averages of all groups compared to increase or decrease from the Vehicle Group was presented. An improvement in small vessels density was observed throughout the study in treated groups (3, 4, 6 and 7M), compared to the vehicle treated group (2M) (FIG. 9).

### Macroscopic Evaluation of Ischemic Severity

The ischemic limb was macroscopically evaluated from Day 7 until Day 35 by using graded morphological scale for necrotic area. Foot amputations were observed in animals from all groups and was lowest in groups 4M and 5M. (See Tables 6 and 7).

TABLE 6
Incidence of Mice with Limb Necrosis Scores 0, land 2 on Day 7

| | Percent of mice | Percent of mice | Percent of mice |
|---|---|---|---|
| | with limb | with limb | with limb |
| | necrosis | necrosis | necrosis |
| Group | score 0 | score 1 | score 2 |
| 2M Vehicle | 64.3 | 35.7 | 0 |
| 3M TI1 | 37.5 | 37.5 | 25.0 |
| 4M TI2 | 36.4 | 63.6 | 0 |
| 5M TI3 | 86.6 | 6.7 | 6.7 |
| 6M TI4 | 64.3 | 35.7 | 0 |
| 7M TI5 | 91.7 | 8.3 | 0 |

TABLE 7
Incidence of Mice with Limb Necrosis Scores 0, 1and 2 on Day 35

| | Percent of mice | Percent of mice | Percent of mice |
|---|---|---|---|
| | with limb | with limb | with limb |
| | necrosis | necrosis | necrosis |
| Group | score 0 | score 1 | score 2 |
| 2M Vehicle | 35.8 | 50.0 | 14.2 |
| 3M TI1 | 25.0 | 25.0 | 50.0 |
| 4M TI2 | 27.3 | 63.6 | 9.1 |
| 5M TI3 | 60.0 | 33.3 | 6.7 |
| 6M TI4 | 23.0 | 38.5 | 38.5 |
| 7M TI5 | 16.7 | 50.0 | 33.3 |

### Assessment of the Limb Functions

Semi-quantitative assessment of impaired use of the ischemic limb was performed from Day 7 until Day 35 by using graded functional scale. A spontaneous improvement in limb function was found in all animal's groups. Nevertheless, animals treated with test items in groups 4M and 5M shoved better functional improvement versus vehicle treated (2M) control group (see Tables 8 and 9).

TABLE 8
Incidence of Mice with Limb Function
Scores 0, 1, 2 and 3 on Day 7

| | Percent | Percent | Percent | Percent |
|---|---|---|---|---|
| | of mice | of mice | of mice | of mice |
| | with limb | with limb | with limb | with limb |
| | function | function | function | function |
| Group | score 0 | score 1 | score 2 | score 3 |
| 2M | 0 | 0 | 71.4 | 28.6 |
| Vehicle | | | | |
| 3M TI1 | 0 | 8.3 | 25.0 | 66.7 |
| 4M TI2 | 0 | 0 | 18.2 | 81.8 |
| 5M TI3 | 0 | 14.3 | 50.0 | 35.7 |
| 6M TI4 | 0 | 7.1 | 78.6 | 14.3 |
| 7M TI5 | 0 | 0 | 66.7 | 33.3 |

TABLE 9
Incidence of mice with limb function
scores 0, 1, 2 and 3 on Day 35

| | Percent | Percent | Percent | Percent |
|---|---|---|---|---|
| | of mice | of mice | of mice | of mice |
| | with limb | with limb | with limb | with limb |
| | function | function | function | function |
| Group | score 0 | score 1 | score 2 | score 3 |
| 2M | 33.3 | 41.7 | 25.0 | 0 |
| Vehicle | | | | |
| 3M TI1 | 44.4 | 44.4 | 11.2 | 0 |
| 4M TI2 | 20.0 | 70.0 | 10.0 | 0 |
| 5M TI3 | 64.3 | 35.7 | 0 | 0 |
| 6M TI4 | 40.0 | 50.0 | 10.0 | 0 |
| 7M TI5 | 25.0 | 37.5 | 37.5 | 0 |

### Histology Results

All slides were stained with H&E and Masson Trichrome staining's and were examined by one pathologist. This evaluation was done as semi-quantitative analysis (see grades below). CD34⁺ high-resolution histology pictures were transferred for quantitative image analysis.

### Muscle Atrophy Grade:

0=There is no atrophy at all.

1=Very mild atrophy (up to 10% of the muscle fibers)

2=mild atrophy (>10% and <25% of the muscles fibers)

3=moderate atrophy (>25% and <75% of the muscles fibers)

4=severe atrophy (>75% and <100% of the muscles fibers)

### Inflammation (Macrophages and Satellite Cells) Grade:

0=There is no inflammatory infiltration at all.

1=Mild cellular infiltration with an increase of up to 10 cells per ×20 HPF.

2=Moderate cellular infiltration with an increase of 10-20 cells per ×20 HPF.

3=High cellular infiltration with an increase of 20-50 cells per ×20 HPF.

4=Very high cellular infiltration with an increase of >50 cells per ×20 HPF.

In all animal groups, a moderate to severe atrophy in the muscles fibers was observed. Degenerative adipose changes of myocytes and an increase of satellite cells and macrophages was observed. In some cases, there was a marked increase of fibrous tissue and lymphocytic infiltrations. Few animals showed some dystrophic mineralization as well. Groups 2M and 3M showed in general more severe change compare to the groups 4M, 5M and 6M. Group 7M showed intermediate change.

### Immunohistochemistry and Analysis of Capillaries Density

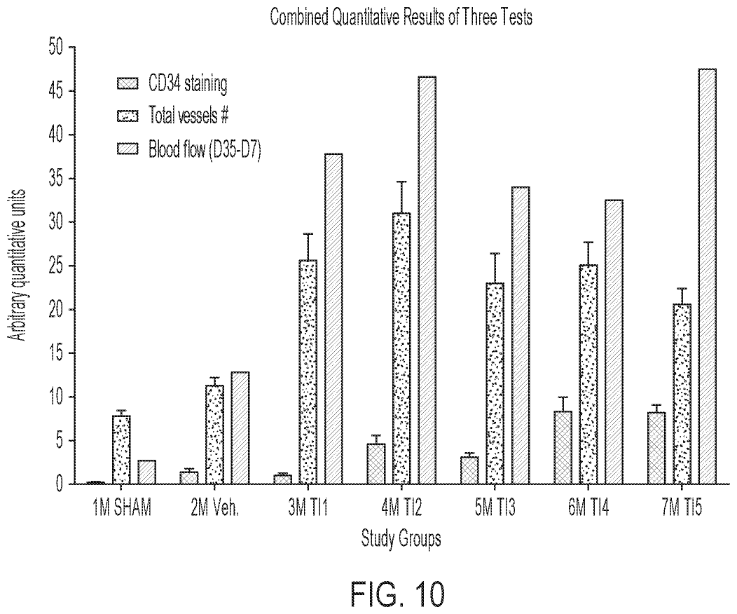

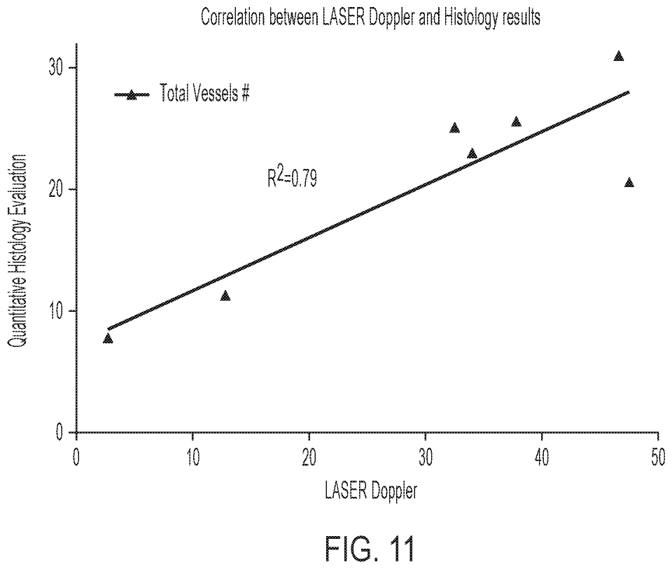

Stained sections were evaluated and photographed by fluorescence microscope (E600; Nikon, Tokyo, Japan) equipped with Plan Fluor objectives connected to a CCD camera (DMX1200F; Nikon). Cy3 shows bright red fluorescence: Ex (max): 543 nm; Em (max): 570 nm while fluorescein Dextran shows intense green fluorescence (Ex(max): 488 nm; Em (max): 530 nM). Digital images were collected and analyzed using Image Pro+ software. Two sections of muscle samples were taken from the same areas in five animals from groups 1M and 7M. The area of blood vessels was measured. Density was expressed as the mean number of capillaries per field of view. Total vessels represent all blood vessels in the measured area. The number of CD-34 positive capillaries was larger in all treated groups compared to the control group 2M on Day 36 of the study. CD-34 positive staining is considered as an indication for small capillaries formation, and thus the obtained results support blood flow improvement observed in the animal groups treated with cells. There was a strong statistically significant correlation between Blood Flow measured by Laser Doppler and Capillaries density (see FIGS. 10 and 11).

### Discussion

IM administration of the Test Items to the ischemic limb revealed some improvement in limb function, primarily in treated groups 4M and 5M, improvement in blood flow (monitored via LASER Doppler), in RSOM imaging and in blood vessels' quantitative histology. The treatments restored blood perfusion by the end of the study (on Day 36) in all treated groups compared to vehicle treated control (in the best group—4M—up to 78% of its normal values). This blood perfusion restoration was well correlated with the results of RSOM imaging analysis and with immunohistochemistry results for capillary density in the operated hind-limb. Rating of the groups indicated 4M as being the best, with 6M and 7M close behind it. STEM 121 staining of gastrocnemius paraffin-embedded slides failed to show human stem-cells, however these were visualized in quadriceps muscles, closer to the injection site.

### Example 10: Bulk Small RNA-Seq Analysis of Meso-3D Vasculonoid VPC2 Cells

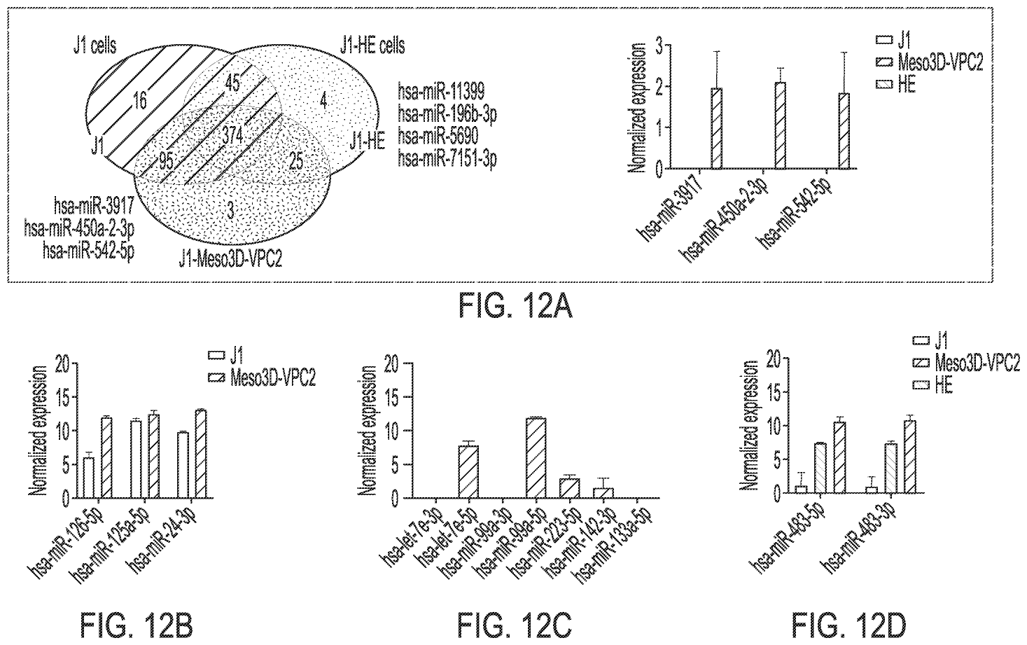

Meso-3D Vasculonoid VPC2 cells were generated according to Example 2. A pellet of about 1-2 million cells was lysed, the RNA isolated, sequenced, bioinformatically aligned, and analyzed for small RNA expression across the known human transcriptome (about 2000 miRNAs). FIG. 12A shows unique human miRNAs found in the population of J1-derived Meso-3D Vasculonoid VPC2 cells from three replicates, including hsa-miR-3917, hsa-miR-450a-2-3p, and hsa-miR-542-5p, as compared to the population of J1 cells and population of J1-derived HE cells. FIG. 12A also shows unique human miRNAs found in the population of J1-derived HE cells, including hsa-miR-11399, hsa-miR-196b-3p, hsa-miR-5690, and hsa-miR-7151-3p.

Additionally, bulk small RNA-seq analysis revealed that miR 214 is expressed at high levels in both J1-derived HE and meso 3D Vasculonoid VPC2 cells and that miR 335-5p is expressed at high levels in J1 and J1-derived HE cells while miR 335-3p is expressed at high levels in J1-derived HE and meso 3D Vasculonoid VPC2 cells. Similarly, miR 199a-3p was expressed at higher levels in both J1-derived HE and meso 3D Vasculonoid VPC2 cells (data not shown).

FIG. 12B shows expression levels of miRNAs in the population of J1-derived Meso-3D Vasculonoid VPC2 cells that were previously analyzed on single cells. FIG. 12B shows that hsa-miR-126-5p, hsa-miR-125a-5p, and hsa-miR-24-3p are expressed in both the population of J1 cells and the population of J1-derived Meso-3D Vasculonoid VPC2 cells. FIG. 12C shows that the population of J1-derived Meso-3D Vasculonoid VPC2 cells expresses hsa-let-7e-5p, hsa-miR-99a-5p, hsa-miR-223-5p, and hsa-miR-142-3p and does not express or has low expression of hsa-let-7e-3p, hsa-miR-99a-3p, and hsa-miR-133a-5p. FIG. 12D also shows that the population of J1-derived Meso-3D Vasculonoid VPC2 cells express hsa-miR-483-5p and hsa-miR-483-3p.

### Example 11: Single Cell RNA-Seq Analysis of Meso-3D Vasculonoid VPC2 Cells

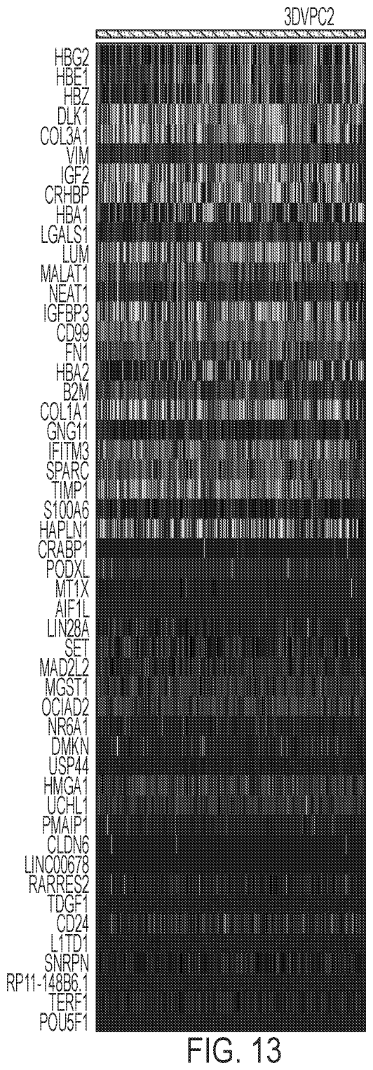

Single cell RNA-seq analysis was also performed on J1-derived Meso-3D Vasculonoid VPC2 cells generated according to Example 2. About 3,700-8,000 single cells for each cell type (J1 cell, J1-derived Meso-3D Vasculonoid VPC2 cell and HUVEC) were captured, processed and analyzed for single cell sequencing using the 10× Genomics (Pleasonton, Calif.) platform and its Cell Ranger analysis pipeline. Further data QC and analyses were performed using the R package Seurat (Butler et al., Nature Biotechnology 36:411-420 (2018); Stuart et al., Cell 177:1888-1902 (2019)). One such analysis was to perform the integrated analysis of J1 cell, J1-derived Meso-3D Vasculonoid VPC2 cell and HUVEC to identify the top differentially expressed genes among the three samples. This analysis followed the guidelines described in Stuart et al., Cell 177:1888-1902 (2019) and also at https://satijalab.org/seurat/v3.0/pancreas_integration_label_transfer.html. Only genes expressed in >10 cells and cells with at least 200 detected genes were retained in the analysis. FIG. 13 shows the expression of the genes most up- or down-regulated in J1-derived Meso-3D Vasculonoid VPC2 cell sample as compared to single J1 or HUVEC cells.

### Example 12: Vasculonoids Exhibit Increased Cell Survival In Vitro and Display Efficacy In Vivo

Vasculonoids of J1-derived Meso-3D Vasculonoid VPC2 cells were generated according to Example 2, but the cells were cryopreserved without dissociating into single cells so that the cells remained in aggregate form.

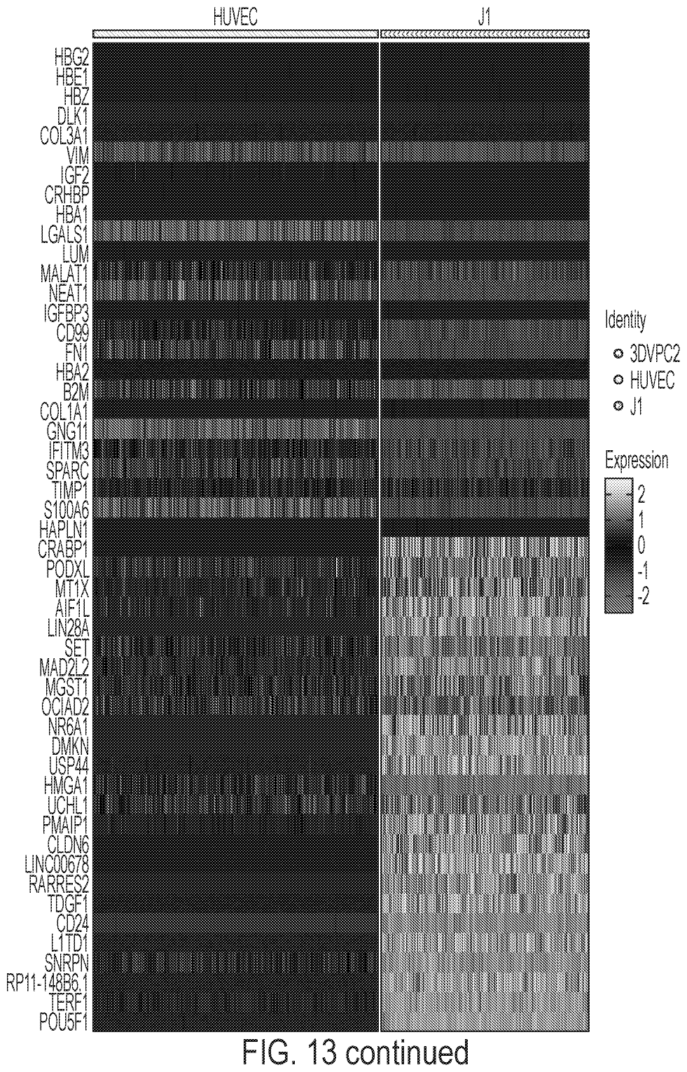

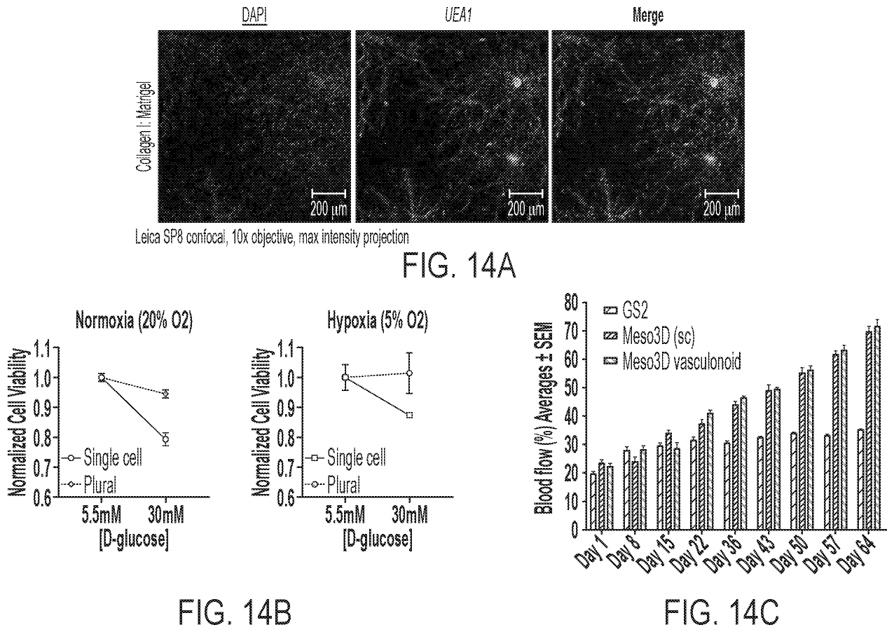

About 150 undissociated Meso-3D Vasculonoid VPC2 (equivalent to about 1,500,000 dissociated single cells) were mixed in a 1:1 ratio of collagen I and growth factor-reduced Matrigel and plated in 4 wells of a 96-well plate. Gels were solidified at 37° C. for 30 min then overlaid with 50 ul complete VascuLife® basal medium (Lifeline® Cell Technology, Frederick, Md.) supplemented with 20 ng/mL FGF, 25 ng/mL BMP4, 45 ng/mL VEGF, and 10 uM SB431542-. Vasculonoids were cultured for 14 days. Gels were fixed with 4% PFA, permeabilized for up to 4 hours with 0.05% Triton-X, and stained with rhodamine conjugated Ulex europaeus I (UEA1), a human specific endothelial cell marker overnight. Gels were thoroughly washed and counterstained with nuclear marker, DAPI. Gels were imaged on a Leica SP8 confocal microscope. FIG. 14A shows at low magnification (10× objective) extensive vascular networks extending from the embedded aggregates of J1-derived Meso-3D Vasculonoid VPC2 vasculonoids after 14 days.

Next, dissociated (or “single”) and undissociated (or vasculonoid or “plural”) Meso-3D Vasculonoid VPC2 cells were seeded into a tissue culture treated 96-well plate (about 14, 000 single cells per well) or ultra-low attachment 96-well plate (about 70 plural cells or vasculonoids per well, equivalent of about 14,000 single cells per well) in 100 ul media. To test the CLI-mimicking conditions (i.e. hyperglycemia and/or hypoxia), cells were cultured with complete VascuLife® basal media (Lifeline® Cell Technology, Frederick, Md.) with 5.5 mM D-glucose as control or with complete VascuLife® basal media with high glucose concentration (30 mM) under either normal (20% O₂) or hypoxic (5% O₂) oxygen conditions for 72 hours. After 72 hours, relative cell survival was measured by incubating each well with 100 ul of CellTiter-Glo® reagent (Promega, Madison, Wis.) for 45 minutes as per manufacturer's directions. Luminescence was measured and normalized to the 5.5 mM control for both single cell and plural cells in each oxygen condition as readout of cell survival. FIG. 14B shows that when these vasculonoids were cultured in the CLI-mimicking conditions in vitro under normoxia (20% O₂) or hypoxia (5% O₂) after thawing as described above, the vasculonoids showed better cell survival compared to J1-derived Meso-3D Vasculonoid VPC2 cells that had been cryopreserved as single cells.

To test for in vivo efficacy, GMP1-Meso3D VPCs were injected either as single cells (Meso3D s.c.; 1 million total single cells per animal) or as undissociated plural cells or vasculonoids (Meso3D vasculonoid; 25,000 per animal, approximately equivalent to 1 million total single cells per animal) into the quadriceps muscle of Balb/c nude mice (n=15 per group) following induction of hindlimb ischemia as detailed in Example 9. Blood flow was assessed by laser Doppler perfusion imaging (LDPI) immediately following surgery and weekly thereafter until day 64. FIG. 14C shows a statistically significant improvement in blood flow after administration of the single cells or vasculonoids throughout the study compared to vehicle treated group (GS2 media only); two-way ANOVA followed by Tukey's test.

### Example 13: Long Term Effect of Meso-3D Vasculonoid VPC2 Cells in the HLI Model

The meso-3D vasculonoid VPC2 cells were generated (as dissociated single cells) according to Example 2 and administered into the HLI animal model described in Example 9 and observed for long term effects. In these studies, 1 million GMP1-derived cells (GMP1 Meso3D vasculonoid VPC2, GMP1-RE, and GMP1-HB) were injected per animal in GS2 media or GS2 media alone (vehicle) into the right quadriceps muscle following HLI surgery (n=12-19 mice/group). Limb necrosis and functional scoring was performed as described in Example 9. For some cell types, more than one lot of cells produced from independent differentiation experiments were used, hence the larger animal count seen when data from more than one lot of the same cell type was combined. Data is mean±sem, averaged across two independent and repeat studies. *p<0.05 vs vehicle control (GS2 media) by one-way ANOVA followed by Dunnett's test.

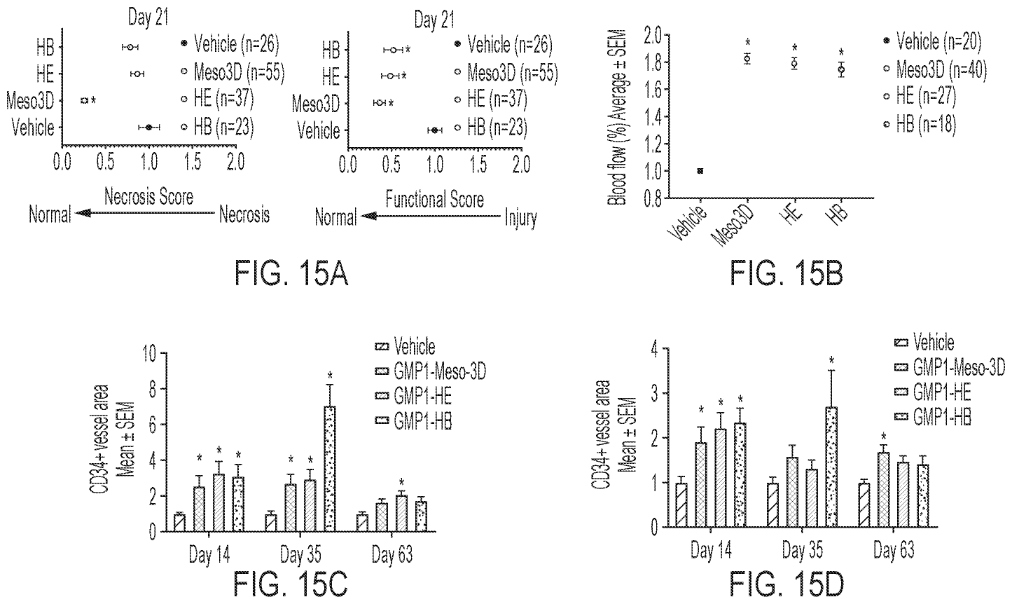

FIG. 15A shows that animals treated with the meso-3D vasculonoid VPC2 cells had better average necrosis and functional scores at Day 21 compared to HE and HB cells. FIG. 15B shows blood flow improvement at Day 63 in animals treated with the meso-3D vasculonoid VPC2 cells, HE, and HB cells, as compared to vehicle. CD34 vessel growth in the quadriceps (FIG. 15C) and in the grastrocnemius (FIG. 15D) showed improvement by all three cell types, with HBs showing better growth at around Day 35. However, by Day 63, all three cell types appeared to promote growth similarly, with the meso-3D vasculonoid VPC2 cells promoting growth slightly better in the gastrocnemius than the HEs and HBs.

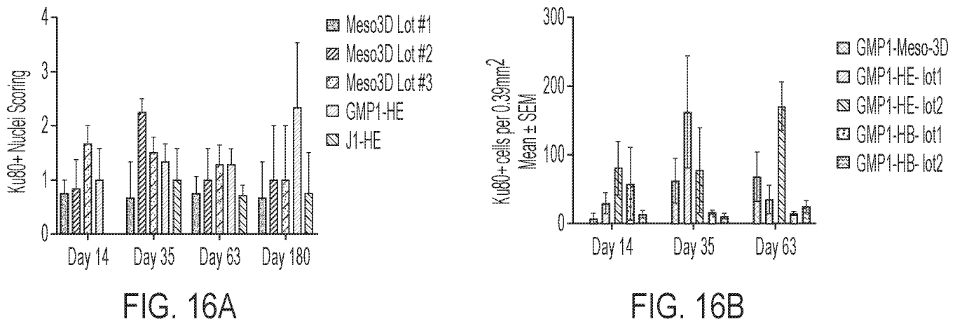

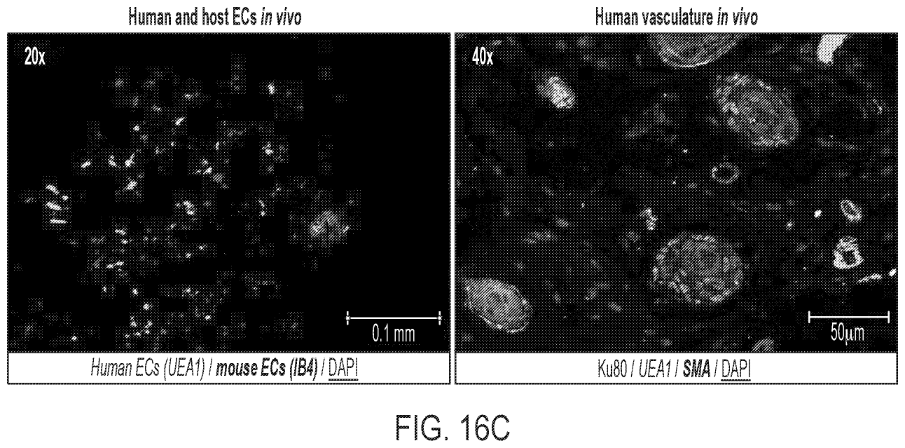

Meso-3D vasculonoid VPC2 cells also showed longer term engraftment beyond 63 days after treatment (FIG. 16A). In this study, 1 million cells per animal in GS2 media or GS2 media alone (vehicle) were injected into the right quadriceps muscle following HLI surgery (Vehicle=18 mice, GMP1-Meso3D Lot #1=19 mice, GMP1-Meso3D Lot #2=18 mice, GMP1-Meso3D Lot #3=19 mice, GMP1-HE=19 mice and J1-RE=19 mice.) The injection site was then marked with a tattoo. At Days 14, 35, 63, and 180, the quadriceps muscle from the operated right hind-limbs were collected, fixed in PFA, embedded in paraffin, and stained for Ku80, a human-specific marker. Two images (20× magnification) were analyzed per animal, with at least n=3 in each group, except J1-RE at Day 14, which only had n=1. Data represents mean±s.e.m according to blinded, independent histopathologist using the following semi-quantitative scale; 0=No positive Ku-80 cells present; 1=<5 positive Ku-80 cells present; 2=>5<15 positive Ku-80 cells present; 3=>15<50 positive Ku-80 cells present; 4=>50 positive Ku-80 cells present. FIG. 16A shows engrafted donor GMP1-Meso3D vasculonoid VPC2 cells at Days 63 and 180 after treatment, indicating long-term engraftment of the cells, although GMP-1-derived HEs appeared to show better engraftment at Day 180.

In a second study (FIG. 16B), 1 million cells per animal in GS2 media were injected into the right quadriceps muscle following HLI surgery (GMP1-Meso3D vasculonoid VPC2 cells=16 mice, GMP1-RE Lot #1=16 mice, GMP1-HE Lot #2=17 mice, GMP1-HB Lot #1=16 mice, GMP1-HB Lot #2=16 mice). The injection site was then marked with a tattoo. At Days 14, 35, and 63, the quadriceps muscle from the operated right hind-limbs were collected, fixed in PFA, embedded in paraffin, and stained for Ku80, a human-specific marker. Two images (20× magnification), taken using an Olympus BX60 light microscope, were analyzed per animal, with at least n=2 in each group. Ku80+ cells were quantified by a blinded, independent histopathologist. Data represents mean±s.e.m. FIG. 16B shows that by Days 35 and 63, the meso-3D vasculonoid VPC2 cells showed engraftment, although one lot of GMP-1-derived HEs appeared to show better engraftment at Day 63.

In another study (FIG. 16C), 1 million cells per animal in GS2 media were injected into the right quadriceps muscle following HLI surgery (GMP1-Meso3D vasculonoid VPC2 cells=24-25 mice from two lots) At Day 63, the quadriceps muscle from the operated right hind-limbs were collected, fixed in PFA and embedded in paraffin. Sections were then stained with either isolectin-B4 (marker of mouse endothelial cells) and Ulex europaeus I (UEA1, marker of human endothelial cells) or Ku80 (pan human specific marker), UEA1, and smooth muscle α-actin (SMA, smooth muscle marker for both mouse and human). DAPI was used to counter-label nuclei. FIG. 16C shows fluorescence images of injected Meso3D vasculonoid VPC2s displaying long-term engraftment (Ku80+), formation of human vasculature (UEA1+ vessels), and promotion of paracrine host vessel growth (IB4+ and SMA+ vessels) 63 days after HLI surgery in Balb/c nude mice.

### EQUIVALENTS

Those skilled in the art will recognize, or be able to ascertain using no more than routine experimentation, many equivalents to the specific embodiments of the invention described herein. such equivalents are intended to be encompassed by the following claims. The contents of all references, patents and published patent applications cited throughout this application are incorporated herein by reference.

1. A method of producing a population of mesoderm-derived vascular progenitor cells (meso-VPCs) from a pluripotent stem cell, wherein the method comprises
culturing a mesoderm cell derived from a pluripotent stem cell under non-adherent or low adherent conditions, in a medium comprising one or more factors selected from the group consisting of vascular endothelial growth factor (VEGF), fibroblast growth factor (FGF), bone morphogenetic protein 4 (BMP4), and a small molecule inhibitor of transforming growth factor-beta (TGF-β) type I receptor, thereby producing a population of mesoderm-derived vascular progenitor cells (meso-VPCs).

2. The method of claim 1, wherein the mesoderm cell is derived from a pluripotent stem cell by culturing the pluripotent stem cell in a medium comprising one or more mesoderm inducing growth factors selected from the group consisting of Activin-A, vascular endothelial growth factor (VEGF), fibroblast growth factor (FGF), and bone morphogenetic protein 4 (BMP4).

3. The method of claim 1, wherein the meso-VPCS are produced as a vasculonoid, optionally, wherein the method further comprises dissociating the meso-VPCs in the vasculonoid into single cells.

4. (canceled)

5. The method of claim 2, wherein the mesoderm inducing growth factors comprise Activin-A, VEGF165, FGF-2 and BMP4, optionally,
wherein the Activin-A is used at a concentration of about 5-15 ng/mL, the VEGF165 is used at a concentration of about 5-25 ng/mL, the FGF-2 is used at a concentration of about 5-25 ng/mL, and/or the BMP4 is used at a concentration of about 5-50 ng/mL.

6. (canceled)

7. (canceled)

8. (canceled)

9. (canceled)

10. The method of claim 2, further comprising removing Activin-A from the culture media after about 24 hours of culturing.

11. The method of claim 2, wherein (a) the pluripotent stem cells are cultured on an extracellular matrix surface, optionally wherein the extracellular matrix surface is a Matrigel-coated surface; and/or (b) the pluripotent stem cells are cultured for about 3 days to about 5 days.

12. (canceled)

13. (canceled)

14. The method of claim 1, wherein (i) the small molecule inhibitor of transforming growth factor-beta (TGF-β) type I receptor is SB431542;
(ii) the one or more factors in step (a) comprise VEGF165, FGF-2, BMP4 and SB431542; optionally, wherein the VEGF165 is used at a concentration of about 10-50 ng/mL, the FGF-2 is used at a concentration of about 10-50 ng/mL, the BMP4 is used at a concentration of about 10-50 ng/mL, and/or the SB431542 is used at a concentration of about 5-20 μM; and/or
(iii) the one or more factors further comprises Forskolin, optionally, wherein the Forskolin is used at a concentration of about 2-10 μM.

15. (canceled)

16. (canceled)

17. (canceled)

18. (canceled)

19. (canceled)

20. (canceled)

21. (canceled)

22. The method of claim 1, wherein culturing the mesoderm cell is (a) performed for about 3 days to about 7 days; (b) conducted under a normoxia condition of 5% CO₂ and 20% O₂; and/or (c) conducted under a normoxia condition of 5% CO₂ and 20% O₂.

23. (canceled)

24. (canceled)

25. The method of claim 1, wherein the non-adherent or low adherent conditions are on an ultra-low attachment surface.

26. A method of producing a population of mesoderm-derived vascular progenitor cell (meso-VPC) from a pluripotent stem cell, wherein the method comprises
(a) culturing a mesoderm cell derived from a pluripotent stem cell on an extracellular matrix surface, in a medium comprising one or more factors selected from the group consisting of vascular endothelial growth factor (VEGF), fibroblast growth factor (FGF), and bone morphogenetic protein 4 (BMP4); and
(b) culturing the cells produced in step (a) on an extracellular matrix surface, in a medium comprising one or more factors selected from the group consisting of vascular endothelial growth factor (VEGF), fibroblast growth factor (FGF), bone morphogenetic protein 4 (BMP4), and a small molecule inhibitor of transforming growth factor-beta (TGF-β) type I receptor,
thereby producing the population of mesoderm-derived vascular progenitor cells.

27. The method of claim 26, wherein the mesoderm cell is derived from a pluripotent stem cell by culturing the pluripotent stem cell in a medium comprising one or more mesoderm inducing growth factors selected from the group consisting of Activin-A, vascular endothelial growth factor (VEGF), fibroblast growth factor (FGF), and bone morphogenetic protein 4 (BMP4).

28. The method of claim 26, wherein the method further comprises dissociating the population of meso-VPCs into single cells.

29. The method of claim 27, wherein the mesoderm inducing growth factors comprise Activin-A, VEGF165, FGF-2 and BMP4, optionally, wherein the Activin-A is used at a concentration of about 5-15 ng/mL, the VEGF165 is used at a concentration of about 5-25 ng/mL, the FGF-2 is used at a concentration of about 5-25 ng/mL, and/or the BMP4 is used at a concentration of about 5-50 ng/mL.

30. (canceled)

31. (canceled)

32. (canceled)

33. (canceled)

34. The method of claim 29, further comprising removing Activin-A from the culture media after about 24 hours of culturing.

35. The method of claim 26, wherein (a) the extracellular matrix surface in step (a) is a collagen IV-coated surface; and/or (b) the pluripotent stem cells are cultured for about 3 days to about 5 days.

36. (canceled)

37. The method of claim 26, wherein
(i) the small molecule inhibitor of transforming growth factor-beta (TGF-β) type I receptor is SB431542; optionally, wherein the SB431542 is used at a concentration of about 5-20 μM;
(ii) the one or more factors in step (a) comprise VEGF165, FGF-2, and BMP4; optionally, wherein the VEGF165 is used at a concentration of about 10-50 ng/mL, the FGF-2 is used at a concentration of about 10-50 ng/mL and/or the BMP4 is used at a concentration of about 10-50 ng/mL;
(iii) the one or more factors in step (b) comprise VEGF165, FGF-2, BMP4 and SB431542; and/or
(iv) the one or more factors in step (a) and/or step (b) further comprises Forskolin, optionally, wherein the Forskolin is used at a concentration of about 2-10 μM.

38. (canceled)

39. (canceled)

40. (canceled)

41. (canceled)

42. (canceled)

43. (canceled)

44. (canceled)

45. (canceled)

46. (canceled)

47. The method of claim 26, wherein
(i) the extracellular matrix surface in steps (a) and (b) is a collagen-IV-coated surface;
(ii) the culturing in step (a) is performed for about 1 day;
(iii) the culturing in step (b) is performed for about 4 days to about 7 days;
(iv) the culturing in step (a) is conducted under a normoxia condition of 5% CO₂ and 20% O₂,
(v) the culturing in step (b) is conducted under a hypoxia condition of 5% CO₂ and 5% O₂, and/or
(vi) culturing of the pluripotent stem cells is conducted under a normoxia condition of 5% CO₂ and 20% O₂.

48. (canceled)

49. (canceled)

50. (canceled)

51. (canceled)

52. (canceled)

53. The method of claim 1, wherein the pluripotent stem cell is a human embryonic stem cell or a human induced pluripotent stem cell.

54. (canceled)

55. The method of claim 1, wherein the population of meso-VPCs
(i) expresses at least one of the cell-surface markers selected from the group consisting of CD31/PECAM1, CD309/KDR, CD43, CD144, CD34, CD184/CXCR4, CD146, and PDGFRb;
(ii) expresses cell-surface markers CD146, CD31/PECAM1, and CD309/KDR;
(iii) expresses CD31/PECAM1, CD309/KDR, CD146, and (a) at least one of CD144, CD34, CD184/CXCR4, CD43, or PDGFRb, (b) CD34, CD184/CXCR4, and PDGFRb, (c) CD184/CXCR4, (d) PDGFRb, (e) CD144 and CD184/CXCR4, (f) CD184/CXCR4 and CD43, or (g) CC184/CXCFR4;
(iv) exhibits limited or no detection of (a) one or more of cell-surface markers selected from the group consisting of CXCR7, CD45, and NG2; (b) CXCR7, CD45, and NG2; or (c) one or more of cell-surface markers selected from the group consisting of CD144, CD34, CD184/CXCR4, CXCR7, CD43, CD45, PDGFRb, and NG2;
(v) expresses at least one miRNA marker selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p;
(vi) exhibits limited or no expression of at least one miRNA marker selected from hsa-let-7e-3p, hsa-miR-99a-3p, hsa-miR-133a-5p, hsa-miR-11399, hsa-miR-196b-3p, hsa-miR-5690, and hsa-miR-7151-3p;
(vii) expresses hsa-miR-3917, hsa-miR-450a-2-3p, and hsa-miR-542-5p;
(viii) comprises at least one meso-VPC positive for at least one miRNA markers selected from the group consisting of mir126, mir125a-5p, mir24, and mir483-5p; and/or
(ix) comprises at least one meso-VPC that exhibits limited or no expression for at least one miRNA markers selected from the group consisting of mir367, mir302a, mir302b, mir302c, mirLet7-e, mir223, mir99a, mir142-3p, and mir133a.

56. (canceled)

57. (canceled)

58. (canceled)

59. (canceled)

60. (canceled)

61. (canceled)

62. (canceled)

63. (canceled)

64. The method of claim 1, further comprising producing a vascular endothelial cell by differentiation of the meso-VPC optionally, wherein the differentiation is performed on a fibronectin-coated surface.

65. (canceled)

66. A composition comprising a population of meso-VPC produced by the method of claim 1.

67. A composition comprising a population of mesoderm-derived vascular progenitor cells (meso-VPCs) produced by in vitro differentiation of a mesoderm cell derived from a pluripotent stem cell, wherein the population of meso-VPCs expresses at least one cell-surface marker selected from the group consisting of CD31/PECAM1, CD309/KDR, CD43, CD144, CD34, CD184/CXCR4, CD146, and PDGFRb.

68. The composition of claim 67, wherein the population of meso-VPCs
(i) expresses at least two cell-surface markers selected from the group consisting of CD31/PECAM1, CD309/KDR, CD43, CD144, CD34, CD184/CXCR4, CD146, and PDGFRb;
(ii) expresses cell surface markers CD146, CD31/PECAM1, and CD309/KDR;
(iii) expresses cell surface markers CD31/PECAM1, CD309/KDR, CD146, and (i) at least one of CD144, CD34, CD184/CXCR4, CD43, or PDGFRb, (ii) CD34, CD184/CXCR4, and PDGFRb, (iii) CD184/CXCR4, (iv) PDGFRb; (v) CD144 and CD184/CXCR4, (vi) CD184/CXCR4 and CD43, or (vii) CC184/CXCFR4;
(iv) exhibits limited or no detection of (a) one or more cell surface markers selected from the group consisting of CXCR7, CD45, and NG2; (b) CXCR7, CD45, and NG2; or (c) one or more cell surface markers selected from the group consisting of CD144, CD34, CD184/CXCR4, CXCR7, CD43, CD45, PDGFRb, and NG2;
(v) expresses at least one miRNA marker selected from hsa-miR-3917, hsa-miR-450a-2-3p, hsa-miR-542-5p, hsa-miR-126-5p, hsa-miR-125a-5p, hsa-miR-24-3p, hsa-let-7e-5p, hsa-miR-99a-5p, hsa-miR-223-5p, hsa-miR-142-3p, hsa-miR-483-5p, hsa-miR-483-3p, miR 214, miR 335-3p, and miR-199a-3p;
(vi) exhibits limited or no expression of at least one miRNA marker selected from hsa-let-7e-3p, hsa-miR-99a-3p, hsa-miR-133a-5p, hsa-miR-11399, hsa-miR-196b-3p, hsa-miR-5690, and hsa-miR-7151-3p; and/or
(vii) expresses hsa-miR-3917, hsa-miR-450a-2-3p, and hsa-miR-542-5p.

69. (canceled)

70. (canceled)

71. (canceled)

72. (canceled)

73. (canceled)

74. (canceled)

75. The composition of claim 67, wherein the population of meso-VPCs comprises (a) vasculonoids of meso-VPCs, or (b) single cells of meso-VPCs.

76. (canceled)

77. A composition comprising a population of meso-VPC produced by in vitro differentiation of a mesoderm cell derived from a pluripotent stem cell, wherein the meso-VPC is positive for at least one miRNA marker selected from the group consisting of mir126, mir125a-5p, mir24, and mir483-5p.

78. The composition of claim 77, wherein the meso-VPC is (i) positive for miRNA marker mir483-5p; and/or (ii) negative for at least one miRNA marker selected from the group consisting of mir367, mir302a, mir302b, mir302c, mirLet7-e, mir223, mir99a, mir142-3p, and mir133a.

79. (canceled)

80. The composition of claim 67, wherein the pluripotent stem cell is a human pluripotent stem cell.

81. The composition of claim 80, wherein the pluripotent stem cell is human embryonic stem cell (hESC) or human induced pluripotent stem cell (hiPSC).

82. (canceled)

83. The composition or meso VPC of claim 67, wherein the pluripotent stem cell is first differentiated into a mesoderm cell which, in turn, is differentiated into the meso-VPC.

84. A pharmaceutical composition comprising the composition or meso VPC of claim 67.

85. A method of treating a vascular disease or disorder in a subject, the method comprising administering to the subject an effective amount of a composition of claim 67, thereby treating the vascular disease or disorder in the subject.

86. The method of claim 85, wherein the vascular disease or disorder is selected from the group consisting of atherosclerosis, peripheral artery disease (PAD), carotid artery disease, venous disease, blood clots, aortic aneurysm, fibromuscular dysplasia, lymphedema, and vascular injury, optionally, wherein the peripheral artery disease is selected from the group consisting of critical limb ischemia, intestinal ischemic syndrome, renal artery disease, popliteal entrapment syndrome, Raynaud's phenomenon, Buerger's disease.

87. (canceled)

88. (canceled)

89. The method of claim 85, wherein the composition, meso-VPC, or the pharmaceutical composition is administered intramuscularly or systemically.

90. The method of claim 85, wherein the administration of the composition, meso-VPC, or the pharmaceutical composition (a) increases the blood flow in the subject; (b) promotes the angiogenesis and/or vasculogenesis in the subject; (c) reduces the ischemic severity in the subject, and/or (d) reduces the necrosis area of the limb in the subject.

91. (canceled)

92. (canceled)

93. (canceled)

94. The method of claim 85, wherein about 1×10⁴ to about 1×10¹³ meso-VPCs are administered to the subject.

95. The method of claim 85, wherein the meso-VPC is administered in a pharmaceutical composition;
wherein the pharmaceutical composition comprises
(a) a buffer, maintaining the solution at a physiological pH;
(b) at least 5% (w/v) glucose; optionally, wherein the glucose is D-glucose (Dextrose) and
(c) an osmotically active agent maintaining the solution at a physiologically osmolality; wherein the osmotically active agent is a salt, optionally, wherein the salt is sodium chloride.

96. (canceled)

97. (canceled)

98. (canceled)

99. (canceled)
